(19) United States
(12) Patent Application Publication
(10) Pub. No.: US 2006/0058326 A1
(43) Pub. Date: Mar. 16, 2006

(54) Pyrimidines, methods for the production thereof, and use thereof

(75) Inventors: Bernd Muller, Frankenthal (DE); Thomas Grote, Wachenheim (DE); Carsten Blettner, Mannheim (DE); Markus Gewehr, Kastellaun (DE); Wassilios Grammenos, Ludwigshafen (DE); Andreas Gypser, Manheim (DE); Joachim Rheinheimer, Ludwigshafen (DE); Peter Schafer, Ottersheim (DE); Frank Schieweck, Hebheim (DE); Anja Schwogler, Manheim (DE); Jordi Tormo I Blasco, Laudenbach (DE); Oliver Wagner, Neustadt (DE); Maria Scherer, Landau (DE); Siegfried Strathmann, Limburgerhof (DE); Ulrich Schofl, Bruhl (DE); Reinhard Stierl, Freinsheim (DE)

Correspondence Address: BIRCH STEWART KOLASCH & BIRCH, PO BOX 747, FALLS CHURCH, VA, 22040-0747, US

(73) Assignee: BASF AKTIENGESLLSCHAFT, LUDWIGSHFAFEN (DE)

(21) Appl. No.: 10/541,386

(22) PCT Filed: Jan. 28, 2004
(86) PCT No.: PCT/EP04/00708

§ 371 Date: Jul. 1, 2005

(30) Foreign Application Priority Data
Feb. 6, 2003 (DE) 103-04-957.6

(51) Int. Cl. A61K 31/513

(52) U.S. Cl. 514/269; 544/315; 544/316

(57) ABSTRACT

Pyrimidines of the formula I 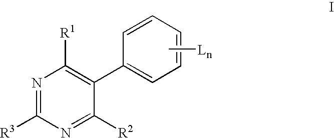
in which Lₙ is as defined in the description and the substituents R¹, R² and R³ are as defined below:
R¹ is C₁-C₁₀-alkyl, C₂-C₁₀-alkenyl, C₂-C₁₀-alkynyl, C₃-C₁₂-cycloalkyl, C₃-C₁₀-cycloalkenyl, phenyl or a five- to ten-membered saturated, partially unsaturated or aromatic heterocycle which is attached via carbon and contains one to four heteroatoms from the group consisting of O, N and S;
R² is halogen, cyano, C₁-C₄-alkyl, C₂-C₄-alkenyl, C₂-C₄-alkynyl, C₁-C₄-alkoxy, C₃-C₄-alkenyloxy or C₃-C₄-alkynyloxy, where the alkyl, alkenyl and alkynyl radicals of R² may be substituted by halogen, cyano, nitro, C₁-C₂-alkoxy or C₁-C₄-alkoxycarbonyl;
R³ is a five- or six-membered saturated, partially unsaturated or aromatic mono- or bicyclic heterocycle which contains one to four heteroatoms from the group consisting of O, N and S; and processes and intermediates for preparing these compounds, compositions comprising them and their use for controlling phytopathogenic harmful fungi are described.

The present invention relates to pyrimidines of the formula I 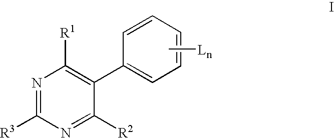
in which the index and the substituents are as defined below:
n is an integer from 1 to 5;
L is halogen, cyano, nitro, cyanato (OCN), C₁-C₈-alkyl, C₂-C₁₀-alkenyl, C₂-C₁₀-alkynyl, C₁-C₆-alkoxy, C₂-C₁₀-alkenyloxy, C₂-C₁₀-alkynyloxy, C₃-C₆-cycloalkyl, C₃-C₆-cycloalkenyl, C₃-C₆-cycloalkoxy, C₃-C₆-cycloalkenyloxy, —C(═O)-A, —C(═O)—O-A, —C(═S)—N(A′)A, —C(═O)—N(A′)A, C(A′)(═N-OA), N(A′)A, N(A′)-C(═O)-A, N(A″)—C(═O)—N(A′)A, S(═O)ₘ-A, S(═O)ₘ—O-A or S(═O)ₘ—N(A′)A;
   m is 0, 1 or 2;
   A,A′, A″ independently of one another are hydrogen, C₀-C₆-alkyl, C₂-C₆-alkenyl, C₂-C₆-alkynyl, C₃-C₈-cycloalkyl, C₃-C₈-cycloalkenyl, where the organic radicals may be partially or fully halogenated or may be substituted by cyano or C₁-C₄-alkoxy, or A and A′ together with the atoms to which they are attached are a five- or six-membered saturated, partially unsaturated or aromatic heterocycle which contains one to four heteroatoms from the group consisting of O, N and S;
   R¹ is C₁-C₁₀-alkyl, C₂-C₁₀-alkenyl, C₂-C₁₀-alkynyl, C₃-C₁₂-cycloalkyl, C₃-C₁₀-cycloalkenyl, phenyl or a five- to ten-membered saturated, partially unsaturated or aromatic heterocycle which is attached via carbon and which contains one to four heteroatoms from the group consisting of O, N and S;
   R² is halogen, cyano, C₁-C₄-alkyl, C₂-C₄-alkenyl, C₂-C₄-alkynyl, C₁-C₄-alkoxy, C₃-C₄-alkenyloxy or C₃-C₄-alkynyloxy;
   R³ is a five- or six-membered saturated, partially unsaturated or aromatic mono- or bicyclic heterocycle which contains one to four heteroatoms from the group consisting of O, N and S,
   where the aliphatic, alicyclic or aromatic groups of the radical definitions of L, R¹, R² and/or R³ for their part may be partially or fully halogenated or may carry one to four groups R^{a}:
      R^{a} is halogen, cyano, C₁-C₈-alkyl, C₂-C₁₀-alkenyl, C₂-C₁₀-alkynyl, C₁-C₆-alkoxy, C₂-C₁₀-alkenyloxy, C₂-C₁₀-alkynyloxy, OH, SH, two vicinal groups R^{a} may be (═O) or (═S), C₃-C₆-cycloalkyl, C₃-C₆-cycloalkenyl, C₃-C₆-cycloalkoxy, C₃-C₆-cycloalkenyloxy, —C(═O)-A, —C(═O)—O-A, —C(═O)—N(A′)A, C(A′)(═N-OA), N(A′)A, N(A′)-C(═O)-A, N(A″)—C(═O)—N(A′)A, S(═O)ₘ-A, S(═O)ₘ—O-A or S(═O)ₘ—N(A′)A, where m, A, A′, A″are as defined above and where the aliphatic, alicyclic or aromatic groups for their part may be partially or fully halogenated or may carry one to three groups R^{b}, where R^{b} has the same meaning as R^{a}.

Moreover, the invention relates to processes and intermediates for preparing these compounds, to the compositions comprising them and to their use for controlling phytopathogenic harmful fungi.

4-Aminopyrimidines having fungicidal action are known from EP-A 407 899 and BE-A 864,399. DE-A 3937284 describes fungicidal 2-pyridyl-4-benzylpyrimidines. WO-A 01/96314 discloses fungicidal pyrimidines which carry a cyanoamino substituent in the 2-position.

However, in many cases their activity is unsatisfactory.

It is an object of the present invention to provide compounds having improved activity.

We have found that this object is achieved by the pyrimidines of the formula I defined at the outset. Moreover, we have found processes and intermediates for their preparation and compositions for controlling harmful fungi, which compositions comprise these compounds.

The compounds I can be obtained by various routes.

The starting materials used can, for example, be the dichloropyrimidines of the formula V whose preparation is described in detail in WO-A O_{2/074753}. In general, the substituent R¹ is initially introduced into the 4-position on the pyrimidine ring by coupling with organometallic reagents (see Scheme 1), giving the compounds of the formula VI.

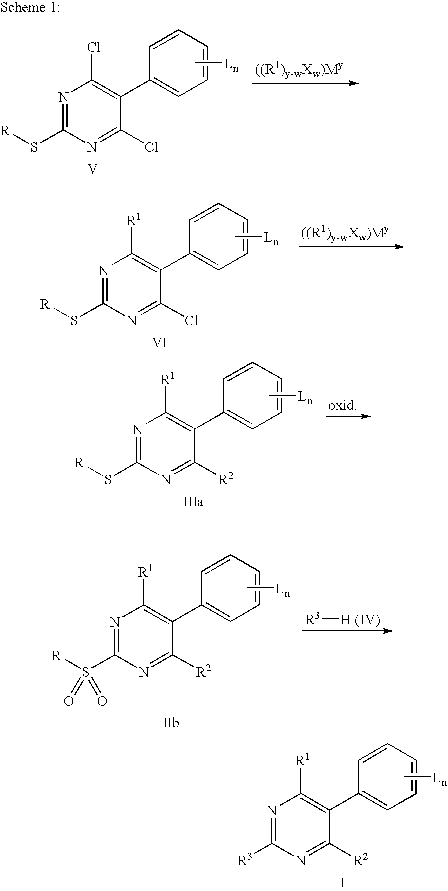

In one embodiment of this process, the reaction is carried out with transition metal catalysis, such as Ni or Pd catalysis. Analogously, the radical R² can be introduced in the 6-position on the pyrimidine ring. In some cases, it may be advisable to change the order and to introduce the substituent R² first.

In the formulae (R¹)_{y-w}X_{w}-M^{y} and (R²)_{y-w}X_{w}-M^{y}, M is a metal ion having the valency Y, such as, for example, B, Zn, Mg, Cu or Sn, X is chlorine, bromine, iodine or hydroxy, R¹ and R² are in particular C₁-C₆-alkyl and w is a number from 0 to 3. This reaction can be carried out, for example, analogously to the following methods: J. Chem. Soc., Perkin Trans. 1 (1994), 1187; ibid. 1 (1996), 2345; WO-A 99/41255; Aust. J. Chem. 43 (1990), 733; J. Org. Chem. 43 (1978), 358; J. Chem. Soc., Chem. Commun. (1979), 866; Tetrahedron Lett. 34 (1993) 8267; ibid. 33 (1992), 413.

What was said above refers in particular to the preparation of compounds in which R² is an alkyl group. If R² is a cyano group or an alkoxy substituent, the radical R² can be introduced by reaction with alkali metal cyanides or alkali metal alkoxides.

Sulfones of the formula IIIb are obtained by oxidizing the corresponding thio compounds IIIa. They are prepared under the conditions disclosed in WO 02/88127. Peracids of organic carboxylic acids or hydrogen peroxide, in particular, have been found to be suitable oxidizing agents. However, the oxidation can also be carried out using, for example, selenium dioxide.

Scheme 2 shows a similar synthesis route as Scheme 1, the only difference being that some synthesis sequences have been changed around. The route shown in Scheme 1 is particularly interesting for preparing the compounds 1′, in which R² is chlorine and for compounds I in which R² is a cyano or alkoxy group.

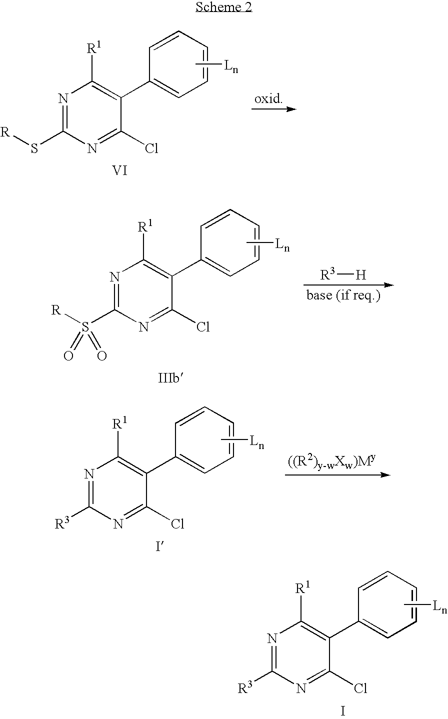

A further advantageous route for preparing the compounds I is shown in Scheme 3. Here, the substituent R^{2′} is a radical attached via carbon, such as alkyl, but not cyano. The pyrimidine ring is constructed by the routes described in WO 97/49697, DD 151404 and JOC 17 (1952), 1320.

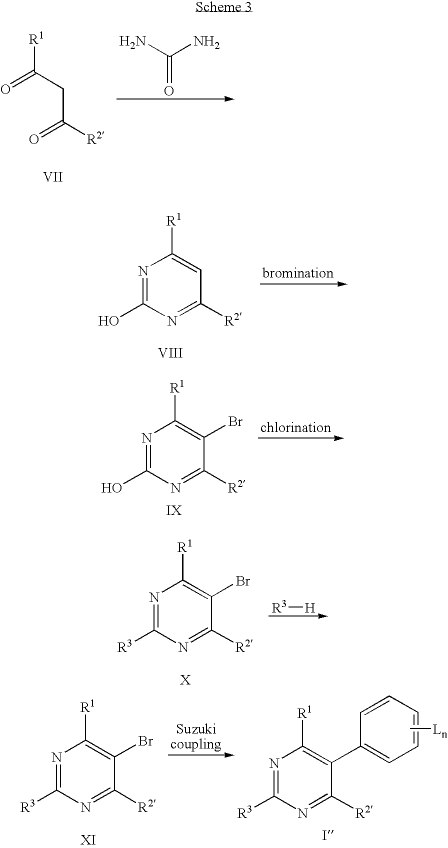

The bromination is preferably carried out using elemental bromine or N-bromosuccinimide. This step can advantageously be carried out in an inert solvent, such as chlorobenzene, nitrobenzene, methylene chloride, chloroform, carbon tetrachloride or a carboxylic acid, such as acetic acid.

Suitable chlorinating agents for converting the hydroxyl compounds IX into the compounds X are, for example, POCl₃, PCl₃/Cl₂ or PCl₅, or mixtures of these reagents. The reaction can be carried out in excess chlorinating agent (POCl₃) or in an inert solvent, such as, for example, acetonitrile, toluene, chlorobenzene or 1,2-dichloroethane. The reaction is preferably carried out in POCl₃.

This conversion is usually carried out at 10 and 180° C. For practical reasons, the reaction temperature usually corresponds to the boiling point of the chlorinating agent used (POCl₃) or of the solvent. The process is advantageously carried out with addition of N,N-dimethylformamide in catalytic or substoichiometric amounts or of nitrogen bases, such as, for example, N,N-dimethylaniline.

In the case of nucleophilic heterocycles, R³ and the pyrimidine ring are preferably linked under the conditions of nucleophilic substitution, usually at 0-200° C., preferably at 10-150° C., in the presence of a dipolar aprotic solvent, such as N,N-dimethyl-formamide, tetrahydrofuran or acetonitrile [cf. DE-A 39 01 084; Chimia 50 (1996), 525-530; Khim. Geterotsikl. Soedin 12 (1998), 1696-1697].

In general, the components are employed in an approximately stoichiometric ratio. However, it may be advantageous to use an excess of the nitrogen heterocycle of the formula R³—H.

In general, the reaction is carried out in the presence of a base, which can be employed in equimolar amounts or else in excess. Suitable bases are alkali metal carbonates and bicarbonates, for example Na₂CO₃ and NaHCO₃, nitrogen bases, such as triethylamine, tributylamine and pyridine, alkali metal alkoxides, such as sodium ethoxide or potassium tert-butoxide, alkali metal amides, such as NaNH₂, or else alkali metal hydrides, such as LiH or NaH.

Moreover, the pyrimidine ring can be linked with the phenyl ring under the reaction conditions of the Suzuki coupling (JOC 67 (2002), 3643; Angew. Chem. 114 (2002), 4350 and literature cited therein).

When constructing the pyrimidine ring, it may be advantageous to introduce the heterocyclyl substituent R³ together with the amidine component, as shown in Scheme 4a. In this case, R^{2′} is again a radical attached via carbon, such as alkyl (but not cyano).

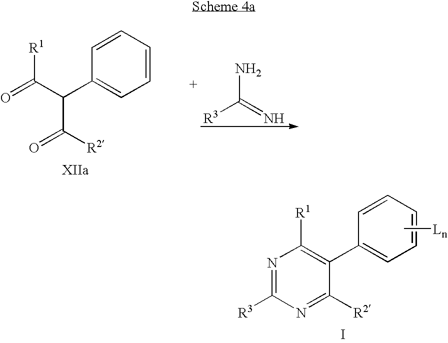

In turn, pyrimidines I, in which R² is halogen or an alkoxy group can advantageously be prepared by the route shown in Scheme 4b. Starting with keto esters XIIb and amidines, the compounds XIII are obtained, which, depending on the nature of the substituent R², can be converted into the respective target compounds 1 or 1′.

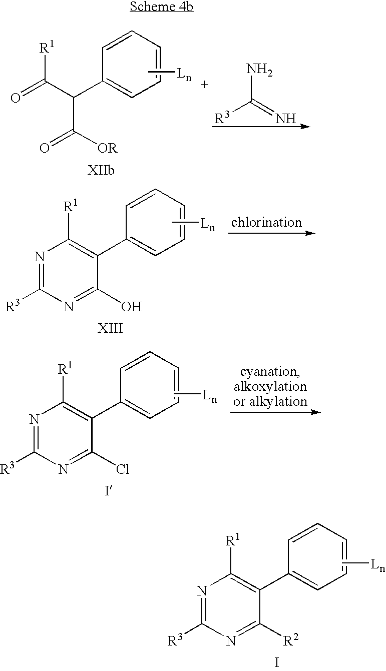

As already mentioned a number of times above, for preparing the pyrimidines I in which R² is a radical attached via carbon, such as alkyl (but not cyano), it is advantageous to use 1,3-dicarbonyl compounds (XIIa) as starting materials. Reaction with urea gives, as shown in Scheme 5, the compounds XIV, which can be chlorinated to give XV.

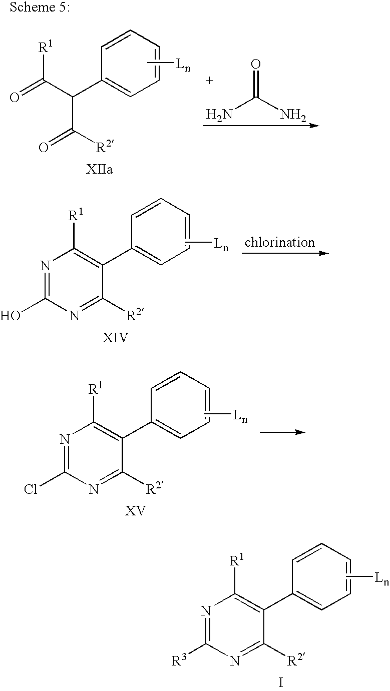

In the case of nucleophilic heterocycles, the substituent R³ is introduced under conditions of nucleophilic substitution.

Moreover, the formation of the bond can also be carried out with transition metal catalysis, such as, for example, under the reaction conditions of the Suzuki coupling.

Furthermore, Scheme 6 shows how the chain of the substituent R¹ can be extended.

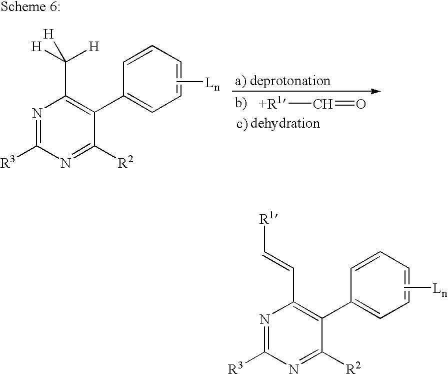

The reaction mixtures are worked up in a customary manner, for example by mixing with water, separating the phases and, if appropriate, chromatographic purification of the crude products. Some of the intermediates and end products are obtained in the form of colorless or slightly brownish viscous oils which can be purified or freed of volatile components under reduced pressure and at moderately elevated temperature. If the intermediates and end products are obtained as solids, purification can also be carried out by recrystallization or digestion.

If individual compounds I are not obtainable by the routes described above, they can be prepared by derivatization of other compounds I.

However, if the synthesis yields isomer mixtures, a separation is generally not necessarily required since in some cases the individual isomers can be converted into one another during the preparation for use or upon use (for example under the action of light, acids or bases). Similar conversions may also occur after use, for example in the treatment of plants in the treated plant or in the harmful fungus to be controlled.

In the definitions of the symbols in the above formulae, collective terms were used which generally represent the following substituents:
halogen: fluorine, chlorine, bromine and iodine;
alkyl: saturated, straight-chain or branched hydrocarbon radicals having 1 to 4, 6, 8 or carbon atoms, for example C₁-C₆-alkyl such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl;
haloalkyl: straight-chain or branched alkyl groups having 1 to 10 carbon atoms (as mentioned above), where some or all of the hydrogen atoms in these groups may be replaced by halogen atoms as mentioned above, for example C₁-C₂-haloalkyl such as chloromethyl, bromomethyl, dichloromethyl, trichloromethyl, fluoromethyl, difluoro-methyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl or 1,1,1-trifluoroprop-2-yl;
alkenyl: unsaturated, straight-chain or branched hydrocarbon radicals having 2 to 4, 6, 8 or 10 carbon atoms and one double bond in any position, for example C₂-C₆-alkenyl such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;
alkadienyl: unsaturated, straight-chain or branched hydrocarbon radicals having 4, 6, 8 or 10 carbon atoms and two double bonds in any position;
haloalkenyl: unsaturated, straight-chain or branched hydrocarbon radicals having 2 to 10 carbon atoms and one double bond in any position (as mentioned above), where some or all of the hydrogen atoms in these groups may be replaced by halogen atoms as mentioned above, in particular by fluorine, chlorine and bromine;
alkynyl: straight-chain or branched hydrocarbon groups having 2 to 4, 6, 8 or 10 carbon atoms and one triple bond in any position, for example C₂-C₆-alkynyl such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;
cycloalkyl: mono- or bicyclic saturated hydrocarbon groups having 3 to 6 or 8 carbon ring members, for example C₃-C₈-cycloalkyl such as cyclopropyl, cyclobutyl, cyclopentyl, cyclohexyl, cycloheptyl and cyclooctyl;
five- to six-membered saturated, partially unsaturated or aromatic heterocycle containing one to four heteroatoms from the group consisting of O, N and S:
   5- or 6-membered heterocyclyl containing one to three nitrogen atoms and/or one oxygen or sulfur atom or one or two oxygen and/or sulfur atoms, for example 2-tetrahydrofuranyl, 3-tetrahydrofuranyl, 2-tetrahydrothienyl, 3-tetrahydrothienyl, 2-pyrrolidynyl, 3-pyrrolidynyl, 3-isoxazolidynyl, 4-isoxazolidynyl, 5-isoxazolidynyl, 3-isothiazolidynyl, 4-isothiazolidynyl, 5-isothiazolidynyl, 3-pyrazolidynyl, 4-pyrazolidynyl, 5-pyrazolidynyl, 2-oxazolidynyl, 4-oxazolidynyl, 5-oxazolidynyl, 2-thiazolidynyl, 4-thiazolidynyl, 5-thiazolidynyl, 2-imidazolidynyl, 4-imidazolidynyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-piperidynyl, 3-piperidynyl, 4-piperidynyl, 1,3-dioxan-5-yl, 2-tetrahydropyranyl, 4-tetrahydropyranyl, 2-tetrahydrothienyl, 3-hexahydropyridazynyl, 4-hexahydropyridazynyl, 2-hexahydropyrimidynyl, 4-hexahydropyrimidynyl, 5-hexahydropyrimidynyl, 2-piperazynyl, 1,3,5-dexahydrotriazin-2-yl and 1,2,4-hexahydrotriazin-3-yl;
   5-membered heteroaryl containing one to four nitrogen atoms or one to three nitrogen atoms and one sulfur or oxygen atom: 5-membered heteroaryl groups which, in addition to carbon atoms, may contain one to four nitrogen atoms or one to three nitrogen atoms and one sulfur or oxygen atom as ring members, for example 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl;
   6-membered heteroaryl containing one to three or one to four nitrogen atoms: 6-membered heteroaryl groups which, in addition to carbon atoms, may contain one to three or one to four nitrogen atoms as ring members, for example 2-pyridynyl, 3-pyridynyl, 4-pyridynyl, 3-pyridazynyl, 4-pyridazynyl, 2-pyrimidynyl, 4-pyrimidynyl, 5-pyrimidynyl, 2-pyrazynyl, 1,3,5-triazin-2-yl and 1,2,4-triazin-3-yl.

The scope of the present invention includes the (R) and (S) isomers and the racemates of compounds of the formula I having chiral centers.

Preferred embodiments of the invention are described below.

Pyrimidines I in which the index and the substituents are as defined below:
L is halogen, cyano, C₁-C₈-alkyl, C₂-C₁₀-alkenyl, C₂-C₁₀-alkynyl, C₁-C₆-alkoxy, C₂-C₁₀-alkenyloxy, C₂-C₁₀-alkynyloxy, —C(═O)—O-A, N(A′)-C(═O)-A or S(═O)ₘ-A;
   m is 0, 1 or 2;

A,A′, A″ independently of one another are hydrogen, C₁-C₆-alkyl, C₂-C₆-alkenyl, C₂-C₆-alkynyl, C₃-C₈-cycloalkyl, where the organic radicals may be partially or fully halogenated or A and A′ together with the atoms to which they are attached are a partially unsaturated or aromatic heterocycle which contains one to four heteroatoms from the group consisting of O, N and S;
R¹ is C₁-C₁₀-alkyl, C₂-C₁₀-alkenyl, C₂-C₁₀-alkynyl, C₃-C₁₂-cycloalkyl, C₃-C₁₀-cycloalkenyl;
R² is C₁-C₄-alkyl, cyano or chlorine,
R³ is as defined above;
where the aliphatic, alicyclic or aromatic groups of the radical definitions of L, R¹ and/or R³ for their part may be partially or fully halogenated or may carry one to four groups R^{a}:
R^{a} is halogen, cyano, C₁-C₈-alkyl, C₂-C₁₀-alkenyl, C₂-C₁₀-alkynyl, C₁-C₆-alkoxy, C₂-C₁₀-alkenyloxy, C₂-C₁₀-alkynyloxy, C₃-C₆-cycloalkyl, C₃-C₆-cycloalkenyl, C₃-C₆-cycloalkoxy, C₃-C₆-cycloalkenyloxy, —C(═O)-A, —C(═O)—O-A, —C(═O)—N(A′)A, C(A′)(═N-OA), N(A′)A, N(A′)-C(═O)-A, N(A″)—C(═O)—N(A′)A, S(═O)ₘ-A, S(═O)ₘ—O-A or S(═O)ₘ—N(A′)A.

With a view to the intended use of the pyrimidines of the formula 1, particular preference is given to the following meanings of the substituents, in each case on their own or in combination:

Preference is given to compounds I in which R¹ is C₃-C₈-alkyl, C₃-C₈-alkenyl, C₃-C₈-alkynyl, C₃-C₆-cycloalkyl or C₅-C₆-cycloalkenyl.

Especially preferred are compounds I in which R¹ is C₁-C₆-alkyl or C₁-C₆-haloalkyl.

In addition, preference is given to compounds I in which R¹ is C₂-C₁₀-alkenyl or C₂-C₁₀-alkynyl.

Likewise, preference is given to compounds I in which R¹ is a 5- or 6-membered saturated or aromatic heterocycle.

Moreover, particular preference is given to compounds I in which R¹ is C₃-C₆-cycloalkyl or C₅-C₆-cycloalkenyl, which radicals may be substituted by C₁-C₄-alkyl or halogen.

Particular preference is given to compounds I in which R^{a} is halogen, cyano, C₁-C₈-alkyl, C₂-C₁₀-alkenyl, C₂-C₁₀-alkynyl, C₁-C₆-alkoxy, C₂-C₁₀-alkenyloxy, C₂-C₁₀-alkynyloxy, C₃-C₆-cycloalkyl, C₃-C₆-cycloalkenyl, C₃-C₆-cycloalkoxy, C₃-C₆-cycloalkenyloxy, —C(═O)-A, —C(═O)—O-A, —C(═O)—N(A′)A, C(A′)(═N-OA), N(A′)A, N(A′)-C(═O)-A, N(A″)—C(═O)—N(A′)A, S(═O)ₘ-A, S(═O)ₘ—OA or S(═O)ₘ—N(A′)A, where the aliphatic or alicyclic groups for their part may be partially or fully halogenated or may carry one to three groups Rb.

Especially preferred are compounds I in which R^{b} is halogen, cyano, C₁-C₆-alkyl, C₂-C₆-alkenyl, C₂-C₆-alkynyl, C₁-C₆-alkylcarbonyl, C₁-C₆-haloalkylcarbonyl, C(A′)(═N-OA) or C₁-C₆-alkoxy.

Particular preference is also given to compounds I in which R² is C₁-C₄-alkyl which may be substituted by halogen.

Likewise, particular preference is given to compounds I in which R² is methyl.

In addition, particular preference is given to compounds I in which R² is halomethyl.

Moreover, particular preference is given to compounds I in which R² is halogen.

Especially preferred are compounds I in which R² is methyl, chlorine or ethyl.

Preference is furthermore given to pyrimidines of the formula I in which R³ is pyrrolyl, pyrazolyl, imidazolyl, 1,2,3-triazolyl, 1,2,4-triazolyl, tetrazolyl, oxazolyl, isoxazolyl, 1,3,4-oxadiazolyl, furanyl, thiophenyl, thiazolyl, isothiazolyl, pyridinyl, pyrimidinyl, pyrazinyl, pyridazinyl, 1,2,3-triazinyl, 1,2,4-triazinyl, pyrrolidinyl, piperidinyl, hexahydroazepinyl or dihydropyridinyl, where the heterocycle may be attached to the pyrimidine ring via C or N and may carry up to three substituents R^{a}.

Especially preferred are pyrimidines of the formula I in which R³ is pyrazol-1-yl, 1,2,4-triazol-1-yl, pyridin-2-yl, pyrimidin-2-yl, pyridazin-3-yl, pyrrolidin-2-on-1-yl, piperidin-2-on-1-yl, hexahydro-2H-azepin-2-on-1-yl, pyrrolidin-2-thion-1-yl, piperidin-2-thion-1-yl, hexahydro-2H-azepin-2-thion-1-yl, 1,2-dihydropyridin-2-on-1-yl.

Preference is given to compounds I in which at least one group L is located ortho to the point of attachment with the pyrimidine skeleton; in particular to those compounds in which n has the value 1, 2 or 3.

Preference is given to pyrimidines I in which Lₙ is halogen, methyl, cyano, ethyl, C₁-haloalkyl, methoxy, —C(═O)-A, —C(═O)—O-A, —C(═O)—N(A′)A, C(A′)(═N-OA), N(A′)-C(═O)-A or S(═O)ₘ-A, where m is 0, 1 or 2 and A, A′ independently of one another are hydrogen or C₁-C₄-alkyl.

Moreover, preference is given to pyrimidines I where the phenyl group substituted by Lₙ is the group B 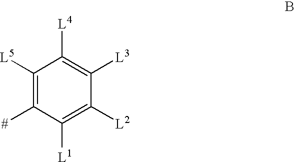
where # is the point of attachment to the pyrimidine skeleton and
L¹ is fluorine, chlorine, CH₃ or CF₃;
L², L⁴ independently of one another are hydrogen, CH₃ or fluorine;
L³ is hydrogen, fluorine, chlorine, bromine, cyano, CH₃, SCH₃, OCH₃, SO₂CH₃, CO—NH₂, CO—NHCH₃, CO—NHC₂H₅, CO—N(CH₃)₂, NH—C(═O)CH₃, N(CH₃)—C(═O)CH₃ or COOCH₃ and
L⁵ is hydrogen, fluorine, chlorine or CH₃.

Moreover, particular preference is given to pyrimidines I where the index and the substituents are as defined below:
L is halogen, cyano, C₁-C₈-alkyl, C₂-C₁₀-alkenyl, C₂-C₁₀-alkynyl, C₁-C₆-alkoxy, C₂-C₁₀-alkenyloxy, C₂-C₁₀-alkynyloxy, C₃-C₆-cycloalkyl, C₃-C₆-cycloalkenyl, C₃-C₆-cycloalkoxy, —C(═O)-A, —C(═O)—O-A, —C(═O)—N(A′)A, C(A′)(═N-OA), N(A′)A, N(A′)-C(═O)-A, N(A″)—C(═O)—N(A′)A, or S(═O)ₘ-A;
m is 0, 1 or 2;
A,A′, A″ independently of one another are hydrogen, C₁-C₆-alkyl, C₂-C₆-alkenyl, C₂-C₆-alkynyl, C₃-C₈-cycloalkyl, C₃-C₈-cycloalkenyl, where the organic radicals may be partially or fully halogenated or may be substituted by cyano or C₁-C₄-alkoxy.

In particular with a view to their use, preference is given to the compounds I compiled in the tables below. Moreover, in their own right, independently of the combination in which they are mentioned, the groups mentioned in the tables for a substituent are a particularly preferred embodiment of the substituent in question. 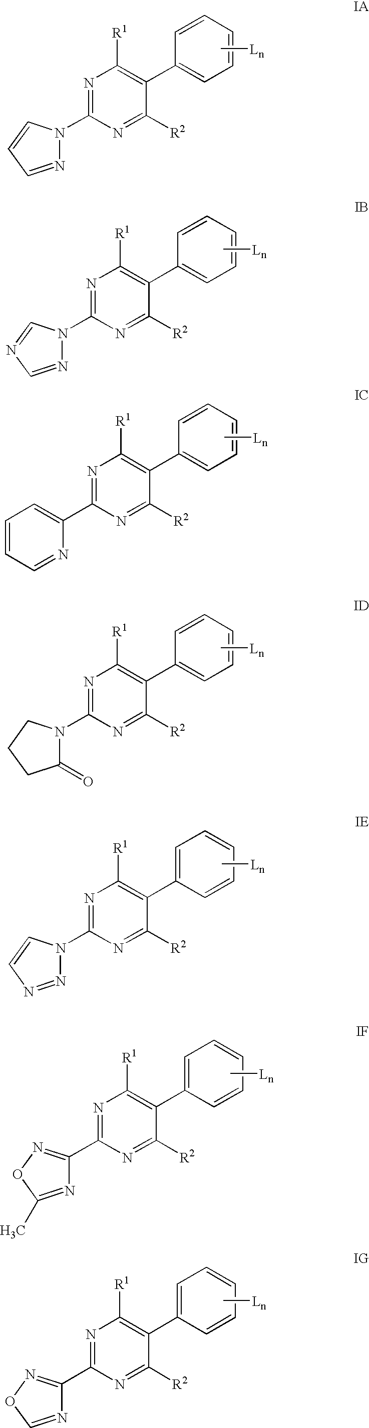 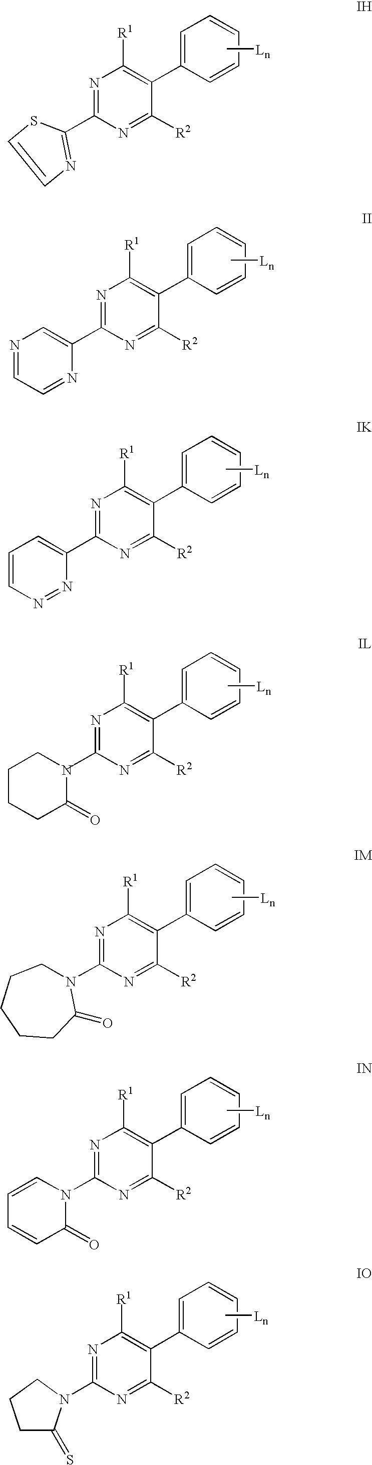 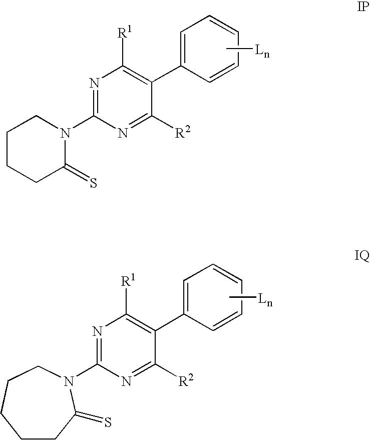
Table 1

Compounds of the formulae IA, IB, IC, ID, IE, IF, IG, IH, II, IK, IL, IM, IN, IO, IP and IQ in which Lₙ is 2-fluoro,6-chloro, R² is methyl and R¹ for a compound corresponds in each case to one row of Table A

Table 2

Compounds of the formulae IA, IB, IC, ID, IE, IF, IG, IH, II, IK, IL, IM, IN, IO, IP and IQ in which Lₙ is 2,6-difluoro, R² is methyl and R¹ for a compound corresponds in each case to one row of Table A

Table 3.

Compounds of the formulae IA, IB, IC, ID, IE, IF, IG, IH, II, IK, IL, IM, IN, IO, IP and IQ in which Lₙ is 2,6-dichloro, R² is methyl and R¹ for a compound corresponds in each case to one row of Table A

Table 4

Compounds of the formulae IA, IB, IC, ID, IE, IF, IG, IH, II, IK, IL, IM, IN, IO, IP and IQ in which Lₙ is 2-fluoro,6-methyl, R² is methyl and R¹ for a compound corresponds in each case to one row of Table A

Table 5

Compounds of the formulae IA, IB, IC, ID, IE, IF, IG, IH, II, IK, IL, IM, IN, IO, IP and IQ in which Lₙ is 2,4,6-trifluoro, R² is methyl and R¹ for a compound corresponds in each case to one row of Table A

Table 6

Compounds of the formulae IA, IB, IC, ID, IE, IF, IG, IH, II, IK, IL, IM, IN, IO, IP and IQ in which Lₙ is 2-methyl,4-fluoro, R² is methyl and R¹ for a compound corresponds in each case to one row of Table A

Table 7

Compounds of the formulae IA, IB, IC, ID, IE, IF, IG, IH, II, IK, IL, IM, IN, IO, IP and IQ in which Lₙ is 2-fluoro,4-methoxycarbonyl, R² is methyl and R¹ for a compound corresponds in each case to one row of Table A

Table 8

Compounds of the formulae IA, IB, IC, ID, IE, IF, IG, IH, II, IK, IL, IM, IN, IO, IP and IQ in which Lₙ is 2-fluoro,4-CN, R² is methyl and R¹ for a compound corresponds in each case to one row of Table A

Table 9

Compounds of the formulae IA, IB, IC, ID, IE, IF, IG, IH, II, IK, IL, IM, IN, IO, IP and IQ in which Lₙ is 2,4,5-trifluoro, R² is methyl and R¹ for a compound corresponds in each case to one row of Table A

Table 10

Compounds of the formulae IA, IB, IC, ID, IE, IF, IG, IH, II, IK, IL, IM, IN, IO, IP and IQ in which Lₙ is 2,4-dichloro, R² is methyl and R¹ for a compound corresponds in each case to one row of Table A

Table 11

Compounds of the formulae IA, IB, IC, ID, IE, IF, IG, IH, II, IK, IL, IM, IN, IO, IP and IQ in which Lₙ is 2-chlorine, R² is methyl and R¹ for a compound corresponds in each case to one row of Table A

Table 12

Compounds of the formulae IA, IB, IC, ID, IE, IF, IG, IH, II, IK, IL, IM, IN, IO, IP and IQ in which Lₙ is 2-fluorine, R² is methyl and R¹ for a compound corresponds in each case to one row of Table A

Table 13

Compounds of the formulae IA, IB, IC, ID, IE, IF, IG, IH, II, IK, IL, IM, IN, IO, IP and IQ in which Lₙ is 2,4-difluoro, R² is methyl and R¹ for a compound corresponds in each case to one row of Table A

Table 14

Compounds of the formulae IA, IB, IC, ID, IE, IF, IG, IH, II, IK, IL, IM, IN, IO, IP and IQ in which Lₙ is 2-fluoro-4-chloro, R² is methyl and R¹ for a compound corresponds in each case to one row of Table A

Table 15

Compounds of the formulae IA, IB, IC, ID, IE, IF, IG, IH, II, IK, IL, IM, IN, IO, IP and IQ in which Lₙ is 2-chloro-4-fluoro, R² is methyl and R¹ for a compound corresponds in each case to one row of Table A

Table 16

Compounds of the formulae IA, IB, IC, ID, IE, IF, IG, IH, II, IK, IL, IM, IN, IO, IP and IQ in which Lₙ is 2,3-difluoro, R² is methyl and R¹ for a compound corresponds in each case to one row of Table A

Table 17

Compounds of the formulae IA, IB, IC, ID, IE, IF, IG, IH, II, IK, IL, IM, IN, IO, IP and IQ in which Lₙ is 2,5-difluoro, R² is methyl and R¹ for a compound corresponds in each case to one row of Table A

Table 18

Compounds of the formulae IA, IB, IC, ID, IE, IF, IG, IH, II, IK, IL, IM, IN, IO, IP and IQ in which Lₙ is 2,3,4-trifluoro, R² is methyl and R¹ for a compound corresponds in each case to one row of Table A

Table 19

Compounds of the formulae IA, IB, IC, ID, IE, IF, IG, IH, II, IK, IL, IM, IN, IO, IP and IQ in which Lₙ is 2-methyl, R² is methyl and R¹ for a compound corresponds in each case to one row of Table A

Table 20

Compounds of the formulae IA, IB, IC, ID, IE, IF, IG, IH, II, IK, IL, IM, IN, IO, IP and IQ in which Lₙ is 2,4-dimethyl, R² is methyl and R¹ for a compound corresponds in each case to one row of Table A

Table 21

Compounds of the formulae IA, IB, IC, ID, IE, IF, IG, IH, II, IK, IL, IM, IN, IO, IP and IQ in which Lₙ is 2-methyl-4-chloro, R² is methyl and R¹ for a compound corresponds in each case to one row of Table A

Table 22

Compounds of the formulae IA, IB, IC, ID, IE, IF, IG, IH, II, IK, IL, IM, IN, IO, IP and IQ in which Lₙ is 2-fluoro-4-methyl, R² is methyl and R¹ for a compound corresponds in each case to one row of Table A

Table 23

Compounds of the formulae IA, IB, IC, ID, IE, IF, IG, IH, II, IK, IL, IM, IN, IO, IP and IQ in which Lₙ is 2,6-dimethyl, R² is methyl and R¹ for a compound corresponds in each case to one row of Table A

Table 24

Compounds of the formulae IA, IB, IC, ID, IE, IF, IG, IH, II, IK, IL, IM, IN, IO, IP and IQ in which Lₙ is 2,4,6-trimethyl, R² is methyl and R¹ for a compound corresponds in each case to one row of Table A

Table 25

Compounds of the formulae IA, IB, IC, ID, IE, IF, IG, IH, II, IK, IL, IM, IN, IO, IP and IQ in which Lₙ is 2,6-difluoro-4-cyano, R² is methyl and R¹ for a compound corresponds in each case to one row of Table A

Table 26

Compounds of the formulae IA, IB, IC, ID, IE, IF, IG, IH, II, IK, IL, IM, IN, IO, IP and IQ in which Lₙ is 2,6-difluoro-4-methyl, R² is methyl and R¹ for a compound corresponds in each case to one row of Table A

Table 27

Compounds of the formulae IA, IB, IC, ID, IE, IF, IG, IH, II, IK, IL, IM, IN, IO, IP and IQ in which Lₙ is 2,6-difluoro-4-methoxycarbonyl, R² is methyl and R¹ for a compound corresponds in each case to one row of Table A

Table 28

Compounds of the formulae IA, IB, IC, ID, IE, IF, IG, IH, II, IK, IL, IM, IN, IO, IP and IQ in which Lₙ is 2-chloro,4-methoxy, R² is methyl and R¹ for a compound corresponds in each case to one row of Table A

Table 29

Compounds of the formulae IA, IB, IC, ID, IE, IF, IG, IH, II, IK, IL, IM, IN, IO, IP and IQ in which Lₙ is 2-chloro,4-methyl, R² is methyl and R¹ for a compound corresponds in each case to one row of Table A

Table 30

Compounds of the formulae IA, IB, IC, ID, IE, IF, IG, IH, II, IK, IL, IM, IN, IO, IP and IQ in which Lₙ is 2-chloro,4-methoxycarbonyl, R² is methyl and R¹ for a compound corresponds in each case to one row of Table A

Table 31

Compounds of the formulae IA, IB, IC, ID, IE, IF, IG, IH, II, IK, IL, IM, IN, IO, IP and IQ in which Lₙ is 2-chloro,4-bromo, R² is methyl and R¹ for a compound corresponds in each case to one row of Table A

Table 32

Compounds of the formulae IA, IB, IC, ID, IE, IF, IG, IH, II, IK, IL, IM, IN, IO, IP and IQ in which Lₙ is 2-chloro,4-cyano, R² is methyl and R¹ for a compound corresponds in each case to one row of Table A

Table 33

Compounds of the formulae IA, IB, IC, ID, IE, IF, IG, IH, II, IK, IL, IM, IN, IO, IP and IQ in which Lₙ is 2,6-difluoro,4-methoxy, R² is methyl and R¹ for a compound corresponds in each case to one row of Table A

Table 34

Compounds of the formulae IA, IB, IC, ID, IE, IF, IG, IH, II, IK, IL, IM, IN, IO, IP and IQ in which Lₙ is 2-fluoro,3-methyl, R² is methyl and R¹ for a compound corresponds in each case to one row of Table A

Table 35

Compounds of the formulae IA, IB, IC, ID, IE, IF, IG, IH, II, IK, IL, IM, IN, IO, IP and IQ in which Lₙ is 2,5-dimethyl, R² is methyl and R¹ for a compound corresponds in each case to one row of Table A

Table 36

Compounds of the formulae IA, IB, IC, ID, IE, IF, IG, IH, II, IK, IL, IM, IN, IO, IP and IQ in which Lₙ is 2-methyl,4-cyano, R² is methyl and R¹ for a compound corresponds in each case to one row of Table A

Table 37

Compounds of the formulae IA, IB, IC, ID, IE, IF, IG, IH, II, IK, IL, IM, IN, IO, IP and IQ in which Lₙ is 2-methyl,4-bromo, R² is methyl and R¹ for a compound corresponds in each case to one row of Table A

Table 38

Compounds of the formulae IA, IB, IC, ID, IE, IF, IG, IH, II, IK, IL, IM, IN, IO, IP and IQ in which Lₙ is 2-methyl,4-fluoro, R² is methyl and R¹ for a compound corresponds in each case to one row of Table A

Table 39

Compounds of the formulae IA, IB, IC, ID, IE, IF, IG, IH, II, IK, IL, IM, IN, IO, IP and IQ in which Lₙ is 2-methyl,4-methoxy, R² is methyl and R¹ for a compound corresponds in each case to one row of Table A

Table 40

Compounds of the formulae IA, IB, IC, ID, IE, IF, IG, IH, II, IK, IL, IM, IN, IO, IP and IQ in which Lₙ is 2-methyl,4-methoxycarbonyl, R² is methyl and R¹ for a compound corresponds in each case to one row of Table A

Table 41

Compounds of the formulae IA, IB, IC, ID, IE, IF, IG, IH, II, IK, IL, IM, IN, IO, IP and IQ in which Lₙ is 2,5-dimethyl,4-bromo, R² is methyl and R¹ for a compound corresponds in each case to one row of Table A

Table 42

Compounds of the formulae IA, IB, IC, ID, IE, IF, IG, IH, II, IK, IL, IM, IN, IO, IP and IQ in which Lₙ is 2-fluoro,4-bromo, R² is methyl and R¹ for a compound corresponds in each case to one row of Table A

Table 43

Compounds of the formulae IA, IB, IC, ID, IE, IF, IG, IH, II, IK, IL, IM, IN, IO, IP and IQ in which Lₙ is 2-fluoro,4-methoxy, R² is methyl and R¹ for a compound corresponds in each case to one row of Table A

Table 44

Compounds of the formulae IA, IB, IC, ID, IE, IF, IG, IH, II, IK, IL, IM, IN, IO, IP and IQ in which Lₙ is 2-fluoro,5-methyl, R² is methyl and R¹ for a compound corresponds in each case to one row of Table A

Table 45

Compounds of the formulae IA, IB, IC, ID, IE, IF, IG, IH, II, IK, IL, IM, IN, IO, IP and IQ in which Lₙ is pentafluoro, R² is methyl and R¹ for a compound corresponds in each case to one row of Table A

Table 46

Compounds of the formulae IA, IB, IC, ID, IE, IF, IG, IH, II, IK, IL, IM, IN, IO, IP and IQ in which Lₙ is 2-fluoro,6-chloro, R² is chloro and R¹ for a compound corresponds in each case to one row of Table A

Table 47

Compounds of the formulae IA, IB, IC, ID, IE, IF, IG, IH, II, IK, IL, IM, IN, IO, IP and IQ in which Lₙ is 2,6-difluoro, R² is chloro and R¹ for a compound corresponds in each case to one row of Table A

Table 48

Compounds of the formulae IA, IB, IC, ID, IE, IF, IG, IH, II, IK, IL, IM, IN, IO, IP and IQ in which L, is 2,6-dichloro, R² is chloro and R¹ for a compound corresponds in each case to one row of Table A

Table 49

Compounds of the formulae IA, IB, IC, ID, IE, IF, IG, IH, II, IK, IL, IM, IN, IO, IP and IQ in which Lₙ is 2-fluoro,6-methyl, R² is chloro and R¹ for a compound corresponds in each case to one row of Table A

Table 50

Compounds of the formulae IA, IB, IC, ID, IE, IF, IG, IH, II, IK, IL, IM, IN, IO, IP and IQ in which Lₙ is 2,4,6-trifluoro, R² is chloro and R¹ for a compound corresponds in each case to one row of Table A

Table 51

Compounds of the formulae IA, IB, IC, ID, IE, IF, IG, IH, II, IK, IL, IM, IN, IO, IP and IQ in which Lₙ is 2-methyl,4-fluoro, R² is chloro and R¹ for a compound corresponds in each case to one row of Table A

Table 52

Compounds of the formulae IA, IB, IC, ID, IE, IF, IG, IH, II, IK, IL, IM, IN, IO, IP and IQ in which Lₙ is 2-fluoro,4-methoxycarbonyl, R² is chloro and R¹ for a compound corresponds in each case to one row of Table A

Table 53

Compounds of the formulae IA, IB, IC, ID, IE, IF, IG, IH, II, IK, IL, IM, IN, IO, IP and IQ in which Lₙ is 2-fluoro,4-CN, R² is chloro and R¹ for a compound corresponds in each case to one row of Table A

Table 54

Compounds of the formulae IA, IB, IC, ID, IE, IF, IG, IH, II, IK, IL, IM, IN, IO, IP and IQ in which Lₙ is 2,4,5-trifluoro, R² is chloro and R¹ for a compound corresponds in each case to one row of Table A

Table 55

Compounds of the formulae IA, IB, IC, ID, IE, IF, IG, IH, II, IK, IL, IM, IN, IO, IP and IQ in which Lₙ is 2,4-dichloro, R² is chloro and R¹ for a compound corresponds in each case to one row of Table A

Table 56

Compounds of the formulae IA, IB, IC, ID, IE, IF, IG, IH, II, IK, IL, IM, IN, IO, IP and IQ in which Lₙ is 2-chloro, R² is chloro and R¹ for a compound corresponds in each case to one row of Table A

Table 57

Compounds of the formulae IA, IB, IC, ID, IE, IF, IG, IH, II, IK, IL, IM, IN, IO, IP and IQ in which Lₙ is 2-fluoro, R² is chloro and R¹ for a compound corresponds in each case to one row of Table A

Table 58

Compounds of the formulae IA, IB, IC, ID, IE, IF, IG, IH, II, IK, IL, IM, IN, IO, IP and IQ in which Lₙ is 2,4-difluoro, R² is chloro and R¹ for a compound corresponds in each case to one row of Table A

Table 59

Compounds of the formulae IA, IB, IC, ID, IE, IF, IG, IH, II, IK, IL, IM, IN, IO, IP and IQ in which Lₙ is 2-fluoro-4-chloro, R² is chloro and R¹ for a compound corresponds in each case to one row of Table A

Table 60

Compounds of the formulae IA, IB, IC, ID, IE, IF, IG, IH, II, IK, IL, IM, IN, IO, IP and IQ in which Lₙ is 2-chloro-4-fluoro, R² is chloro and R¹ for a compound corresponds in each case to one row of Table A

Table 61

Compounds of the formulae IA, IB, IC, ID, IE, IF, IG, IH, II, IK, IL, IM, IN, IO, IP and IQ in which Lₙ is 2,3-difluoro, R² is chloro and R¹ for a compound corresponds in each case to one row of Table A

Table 62

Compounds of the formulae IA, IB, IC, ID, IE, IF, IG, IH, II, IK, IL, IM, IN, IO, IP and IQ in which Lₙ is 2,5-difluoro, R² is chloro and R¹ for a compound corresponds in each case to one row of Table A

Table 63

Compounds of the formulae IA, IB, IC, ID, IE, IF, IG, IH, II, IK, IL, IM, IN, IO, IP and IQ in which Lₙ is 2,3,4-trifluoro, R² is chloro and R¹ for a compound corresponds in each case to one row of Table A

Table 64

Compounds of the formulae IA, IB, IC, ID, IE, IF, IG, IH, II, IK, IL, IM, IN, IO, IP and IQ in which Lₙ is 2-methyl, R² is chloro and R¹ for a compound corresponds in each case to one row of Table A

Table 65

Compounds of the formulae IA, IB, IC, ID, IE, IF, IG, IH, II, IK, IL, IM, IN, IO, IP and IQ in which Lₙ is 2,4-dimethyl, R² is chloro and R¹ for a compound corresponds in each case to one row of Table A

Table 66

Compounds of the formulae IA, IB, IC, ID, IE, IF, IG, IH, II, IK, IL, IM, IN, IO, IP and IQ in which Lₙ is 2-methyl-4-chloro, R² is chloro and R¹ for a compound corresponds in each case to one row of Table A

Table 67

Compounds of the formulae IA, IB, IC, ID, IE, IF, IG, IH, II, IK, IL, IM, IN, IO, IP and IQ in which Lₙ is 2-fluoro-4-methyl, R² is chloro and R¹ for a compound corresponds in each case to one row of Table A

Table 68

Compounds of the formulae IA, IB, IC, ID, IE, IF, IG, IH, II, IK, IL, IM, IN, IO, IP and IQ in which Lₙ is 2,6-dimethyl, R² is chloro and R¹ for a compound corresponds in each case to one row of Table A

Table 69

Compounds of the formulae IA, IB, IC, ID, IE, IF, IG, IH, II, IK, IL, IM, IN, IO, IP and IQ in which Lₙ is 2,4,6-trimethyl, R² is chloro and R¹ for a compound corresponds in each case to one row of Table A

Table 70

Compounds of the formulae IA, IB, IC, ID, IE, IF, IG, IH, II, IK, IL, IM, IN, IO, IP and IQ in which Lₙ is 2,6-difluoro-4-cyano, R² is chloro and R¹ for a compound corresponds in each case to one row of Table A

Table 71

Compounds of the formulae IA, IB, IC, ID, IE, IF, IG, IH, II, IK, IL, IM, IN, IO, IP and IQ in which Lₙ is 2,6-difluoro-4-methyl, R² is chloro and R¹ for a compound corresponds in each case to one row of Table A

Table 72

Compounds of the formulae IA, IB, IC, ID, IE, IF, IG, IH, II, IK, IL, IM, IN, IO, IP and IQ in which Lₙ is 2,6-difluoro-4-methoxycarbonyl, R² is chloro and R¹ for a compound corresponds in each case to one row of Table A

Table 73

Compounds of the formulae IA, IB, IC, ID, IE, IF, IG, IH, II, IK, IL, IM, IN, IO, IP and IQ in which Lₙ is 2-chloro,4-methoxy, R² is chloro and R¹ for a compound corresponds in each case to one row of Table A

Table 74

Compounds of the formulae IA, IB, IC, ID, IE, IF, IG, IH, II, IK, IL, IM, IN, IO, IP and IQ in which Lₙ is 2-chloro,4-methyl, R² is chloro and R¹ for a compound corresponds in each case to one row of Table A

Table 75

Compounds of the formulae IA, IB, IC, ID, IE, IF, IG, IH, II, IK, IL, IM, IN, IO, IP and IQ in which Lₙ is 2-chloro,4-methoxycarbonyl, R² is chloro and R¹ for a compound corresponds in each case to one row of Table A

Table 76

Compounds of the formulae IA, IB, IC, ID, IE, IF, IG, IH, II, IK, IL, IM, IN, IO, IP and IQ in which Lₙ is 2-chloro,4-bromo, R² is chloro and R¹ for a compound corresponds in each case to one row of Table A

Table 77

Compounds of the formulae IA, IB, IC, ID, IE, IF, IG, IH, II, IK, IL, IM, IN, IO, IP and IQ in which Lₙ is 2-chloro,4-cyano, R² is chloro and R¹ for a compound corresponds in each case to one row of Table A

Table 78

Compounds of the formulae IA, IB, IC, ID, IE, IF, IG, IH, II, IK, IL, IM, IN, IO, IP and IQ in which Lₙ is 2,6-difluoro,4-methoxy, R² is chloro and R¹ for a compound corresponds in each case to one row of Table A

Table 79

Compounds of the formulae IA, IB, IC, ID, IE, IF, IG, IH, II, IK, IL, IM, IN, IO, IP and IQ in which Lₙ is 2-fluoro,3-methyl, R² is chloro and R¹ for a compound corresponds in each case to one row of Table A

Table 80

Compounds of the formulae IA, IB, IC, ID, IE, IF, IG, IH, II, IK, IL, IM, IN, IO, IP and IQ in which Lₙ is 2,5-dimethyl, R² is chloro and R¹ for a compound corresponds in each case to one row of Table A

Table 81

Compounds of the formulae IA, IB, IC, ID, IE, IF, IG, IH, II, IK, IL, IM, IN, IO, IP and IQ in which Lₙ is 2-methyl,4-cyano, R² is chloro and R¹ for a compound corresponds in each case to one row of Table A

Table 82

Compounds of the formulae IA, IB, IC, ID, IE, IF, IG, IH, II, IK, IL, IM, IN, IO, IP and IQ in which Lₙ is 2-methyl,4-bromo, R² is chloro and R¹ for a compound corresponds in each case to one row of Table A

Table 83

Compounds of the formulae IA, IB, IC, ID, IE, IF, IG, IH, II, IK, IL, IM, IN, IO, IP and IQ in which Lₙ is 2-methyl,4-fluoro, R² is chloro and R¹ for a compound corresponds in each case to one row of Table A

Table 84

Compounds of the formulae IA, IB, IC, ID, IE, IF, IG, IH, II, IK, IL, IM, IN, IO, IP and IQ in which Lₙ is 2-methyl,4-methoxy, —R² is chloro and R¹ for a compound corresponds in each case to one row of Table A

Table 85

Compounds of the formulae IA, IB, IC, lb, IE, IF, IG, IH, II, IK, IL, IM, IN, IO, IP and IQ in which Lₙ is 2-methyl,4-methoxycarbonyl, R² is chloro and R¹ for a compound corresponds in each case to one row of Table A

Table 86

Compounds of the formulae IA, IB, IC, ID, IE, IF, IG, IH, II, IK, IL, IM, IN, IO, IP and IQ in which Lₙ is 2,5-dimethyl,4-bromo, R² is chloro and R¹ for a compound corresponds in each case to one row of Table A

Table 87

Compounds of the formulae IA, IB, IC, ID, IE, IF, IG, IH, II, IK, IL, IM, IN, IO, IP and IQ in which Lₙ is 2-fluoro,4-bromo, R² is chloro and R¹ for a compound corresponds in each case to one row of Table A

Table 88

Compounds of the formulae IA, IB, IC, ID, IE, IF, IG, IH, II, IK, IL, IM, IN, IO, IP and IQ in which Lₙ is 2-fluoro,4-methoxy, R² is chloro and R¹ for a compound corresponds in each case to one row of Table A

Table 89

Compounds of the formulae IA, IB, IC, ID, IE, IF, IG, IH, II, IK, IL, IM, IN, IO, IP and IQ in which Lₙ is 2-fluoro,5-methyl, R² is chloro and R¹ for a compound corresponds in each case to one row of Table A

Table 90

Compounds of the formulae IA, IB, IC, ID, IE, IF, IG, IH, II, IK, IL, IM, IN, IO, IP and IQ in which Lₙ is pentafluoro, R² is chloro and R¹ for a compound corresponds in each case to one row of Table A

Table 91

Compounds of the formulae IA, IB, IC, ID, IE, IF, IG, IH, II, IK, IL, IM, IN, IO, IP and IQ in which Lₙ is 2-fluoro,6-chloro, R² is methoxy and R¹ for a compound corresponds in each case to one row of Table A

Table 92

Compounds of the formulae IA, IB, IC, ID, IE, IF, IG, IH, II, IK, IL, IM, IN, IO, IP and IQ in which Lₙ is 2,6-difluoro, R² is methoxy and R¹ for a compound corresponds in each case to one row of Table A

Table 93

Compounds of the formulae IA, IB, IC, ID, IE, IF, IG, IH, II, IK, IL, IM, IN, IO, IP and IQ in which Lₙ is 2,6-dichloro, R² is methoxy and R¹ for a compound corresponds in each case to one row of Table A

Table 94

Compounds of the formulae IA, IB, IC, ID, IE, IF, IG, IH, II, IK, IL, IM, IN, IO, IP and IQ in which Lₙ is 2-fluoro,6-methyl, R² is methoxy and R¹ for a compound corresponds in each case to one row of Table A

Table 95

Compounds of the formulae IA, IB, IC, ID, IE, IF, IG, IH, II, IK, IL, IM, IN, IO, IP and IQ in which Lₙ is 2,4,6-trifluoro, R² is methoxy and R¹ for a compound corresponds in each case to one row of Table A

Table 96

Compounds of the formulae IA, IB, IC, ID, IE, IF, IG, IH, II, IK, IL, IM, IN, IO, IP and IQ in which Lₙ is 2-methyl,4-fluoro, R² is methoxy and R¹ for a compound corresponds in each case to one row of Table A

Table 97

Compounds of the formulae IA, IB, IC, ID, IE, IF, IG, IH, II, IK, IL, IM, IN, IO, IP and IQ in which Lₙ is 2-fluoro,4-methoxycarbonyl, R² is methoxy and R¹ for a compound corresponds in each case to one row of Table A

Table 98

Compounds of the formulae IA, IB, IC, ID, IE, IF, IG, IH, II, IK, IL, IM, IN, IO, IP and IQ in which Lₙ is 2-fluoro,4-CN, R² is methoxy and R¹ for a compound corresponds in each case to one row of Table A

Table 99

Compounds of the formulae IA, IB, IC, ID, IE, IF, IG, IH, II, IK, IL, IM, IN, IO, IP and IQ in which Lₙ is 2,4,5-trifluoro, R² is methoxy and R¹ for a compound corresponds in each case to one row of Table A

Table 100

Compounds of the formulae IA, IB, IC, ID, IE, IF, IG, IH, II, IK, IL, IM, IN, IO, IP and IQ in which Lₙ is 2,4-dichloro, R² is methoxy and R¹ for a compound corresponds in each case to one row of Table A

Table 101

Compounds of the formulae IA, IB, IC, ID, IE, IF, IG, IH, II, IK, IL, IM, IN, IO, IP and IQ in which Lₙ is 2-chloro, R² is methoxy and R¹ for a compound corresponds in each case to one row of Table A

Table 102

Compounds of the formulae IA, IB, IC, ID, IE, IF, IG, IH, II, IK, IL, IM, IN, IO, IP and IQ in which Lₙ is 2-fluoro, R² is methoxy and R¹ for a compound corresponds in each case to one row of Table A

Table 103

Compounds of the formulae IA, IB, IC, ID, IE, IF, IG, IH, II, IK, IL, IM, IN, IO, IP and IQ in which Lₙ is 2,4-difluoro, R² is methoxy and R¹ for a compound corresponds in each case to one row of Table A

Table 104

Compounds of the formulae IA, IB, IC, ID, IE, IF, IG, IH, II, IK, IL, IM, IN, IO, IP and IQ in which Lₙ is 2-fluoro-4-chloro, R² is methoxy and R¹ for a compound corresponds in each case to one row of Table A

Table 105

Compounds of the formulae IA, IB, IC, ID, IE, IF, IG, IH, II, IK, IL, IM, IN, IO, IP and IQ in which Lₙ is 2-chloro-4-fluoro, R² is methoxy and R¹ for a compound corresponds in each case to one row of Table A

Table 106

Compounds of the formulae IA, IB, IC, ID, IE, IF, IG, IH, II, IK, IL, IM, IN, IO, IP and IQ in which Lₙ is 2,3-difluoro, R² is methoxy and R¹ for a compound corresponds in each case to one row of Table A

Table 107

Compounds of the formulae IA, IB, IC, ID, IE, IF, IG, IH, II, IK, IL, IM, IN, IO, IP and IQ in which Lₙ is 2,5-difluoro, R² is methoxy and R¹ for a compound corresponds in each case to one row of Table A

Table 108

Compounds of the formulae IA, IB, IC, ID, IE, IF, IG, IH, II, IK, IL, IM, IN, IO, IP and IQ in which Lₙ is 2,3,4-trifluoro, —R² is methoxy and R¹ for a compound corresponds in each case to one row of Table A

Table 109

Compounds of the formulae IA, IB, IC, ID, IE, IF, IG, IH, II, IK, IL, IM, IN, IO, IP and IQ in which Lₙ is 2-methyl, R² is methoxy and R¹ for a compound corresponds in each case to one row of Table A

Table 110

Compounds of the formulae IA, IB, IC, ID, IE, IF, IG, IH, II, IK, IL, IM, IN, IO, IP and IQ in which Lₙ is 2,4-dimethyl, R² is methoxy and R¹ for a compound corresponds in each case to one row of Table A

Table 111

Compounds of the formulae IA, IB, IC, ID, IE, IF, IG, IH, II, IK, IL, IM, IN, IO, IP and IQ in which Lₙ is 2-methyl-4-chloro, R² is methoxy and R¹ for a compound corresponds in each case to one row of Table A

Table 112

Compounds of the formulae IA, IB, IC, ID, IE, IF, IG, IH, II, IK, IL, IM, IN, IO, IP and IQ in which Lₙ is 2-fluoro-4-methyl, R² is methoxy and R¹ for a compound corresponds in each case to one row of Table A

Table 113

Compounds of the formulae IA, IB, IC, ID, IE, IF, IG, IH, II, IK, IL, IM, IN, IO, IP and IQ in which Lₙ is 2,6-dimethyl, R² is methoxy and R¹ for a compound corresponds in each case to one row of Table A

Table 114

Compounds of the formulae IA, IB, IC, ID, IE, IF, IG, IH, II, IK, IL, IM, IN, IO, IP and IQ in which Lₙ is 2,4,6-trimethyl, R² is methoxy and R¹ for a compound corresponds in each case to one row of Table A

Table 115

Compounds of the formulae IA, IB, IC, ID, IE, IF, IG, IH, II, IK, IL, IM, IN, IO, IP and IQ in which Lₙ is 2,6-difluoro-4-cyano, R² is methoxy and R¹ for a compound corresponds in each case to one row of Table A

Table 116

Compounds of the formulae IA, IB, IC, ID, IE, IF, IG, IH, II, IK, IL, IM, IN, IO, IP and IQ in which Lₙ is 2,6-difluoro-4-methyl, R² is methoxy and R¹ for a compound corresponds in each case to one row of Table A

Table 117

Compounds of the formulae IA, IB, IC, ID, IE, IF, IG, IH, II, IK, IL, IM, IN, IO, IP and IQ in which Lₙ is 2,6-difluoro-4-methoxycarbonyl, R² is methoxy and R¹ for a compound corresponds in each case to one row of Table A

Table 118

Compounds of the formulae IA, IB, IC, ID, IE, IF, IG, IH, II, IK, IL, IM, IN, IO, IP and IQ in which Lₙ is 2-chloro,4-methoxy, R² is methoxy and R¹ for a compound corresponds in each case to one row of Table A

Table 119

Compounds of the formulae IA, IB, IC, ID, IE, IF, IG, IH, II, IK, IL, IM, IN, IO, IP and IQ in which Lₙ is 2-chloro,4-methyl, R² is methoxy and R¹ for a compound corresponds in each case to one row of Table A

Table 120

Compounds of the formulae IA, IB, IC, ID, IE, IF, IG, IH, II, IK, IL, IM, IN, IO, IP and IQ in which Lₙ is 2-chloro,4-methoxycarbonyl, R² is methoxy and R¹ for a compound corresponds in each case to one row of Table A

Table 121

Compounds of the formulae IA, IB, IC, ID, IE, IF, IG, IH, II, IK, IL, IM, IN, IO, IP and IQ in which Lₙ is 2-chloro,4-bromo, R² is methoxy and R¹ for a compound corresponds in each case to one row of Table A

Table 122

Compounds of the formulae IA, IB, IC, ID, IE, IF, IG, IH, II, IK, IL, IM, IN, IO, IP and IQ in which Lₙ is 2-chloro,4-cyano, R² is methoxy and R¹ for a compound corresponds in each case to one row of Table A

Table 123

Compounds of the formulae IA, IB, IC, ID, IE, IF, IG, IH, II, IK, IL, IM, IN, IO, IP and IQ in which Lₙ is 2,6-difluoro,4-methoxy, R² is methoxy and R¹ for a compound corresponds in each case to one row of Table A

Table 124

Compounds of the formulae IA, IB, IC, ID, IE, IF, IG, IH, II, IK, IL, IM, IN, IO, IP and IO in which Lₙ is 2-fluoro,3-methyl, R² is methoxy and R¹ for a compound corresponds in each case to one row of Table A

Table 125

Compounds of the formulae IA, IB, IC, ID, IE, IF, IG, IH, II, IK, IL, IM, IN, IO, IP and IQ in which Lₙ is 2,5-dimethyl, R² is methoxy and R¹ for a compound corresponds in each case to one row of Table A

Table 126

Compounds of the formulae IA, IB, IC, ID, IE, IF, IG, IH, II, IK, IL, IM, IN, IO, IP and IQ in which Lₙ is 2-methyl,4-cyano, R² is methoxy and R¹ for a compound corresponds in each case to one row of Table A

Table 127

Compounds of the formulae IA, IB, IC, ID, IE, IF, IG, IH, II, IK, IL, IM, IN, IO, IP and IQ in which Lₙ is 2-methyl,4-bromo, R² is methoxy and R¹ for a compound corresponds in each case to one row of Table A

Table 128

Compounds of the formulae IA, IB, IC, ID, IE, IF, IG, IH, II, IK, IL, IM, IN, IO, IP and IQ in which Lₙ is 2-methyl,5-fluoro, R² is methoxy and R¹ for a compound corresponds in each case to one row of Table A

Table 129

Compounds of the formulae IA, IB, IC, ID, IE, IF, IG, IH, II, IK, IL, IM, IN, IO, IP and IQ in which Lₙ is 2-methyl,4-methoxy, R² is methoxy and R¹ for a compound corresponds in each case to one row of Table A

Table 130

Compounds of the formulae IA, IB, IC, ID, IE, IF, IG, IH, II, IK, IL, IM, IN, IO, IP and IQ in which Lₙ is 2-methyl,4-methoxycarbonyl, R² is methoxy and R¹ for a compound corresponds in each case to one row of Table A

Table 131

Compounds of the formulae IA, IB, IC, ID, IE, IF, IG, IH, II, IK, IL, IM, IN, IO, IP and IQ in which Lₙ is 2,5-dimethyl,4-bromo, R² is methoxy and R¹ for a compound corresponds in each case to one row of Table A

Table 132

Compounds of the formulae IA, IB, IC, ID, IE, IF, IG, IH, II, IK, IL, IM, IN, IO, IP and IQ in which Lₙ is 2-fluoro,4-bromo, R² is methoxy and R¹ for a compound corresponds in each case to one row of Table A

Table 133

Compounds of the formulae IA, IB, IC, ID, IE, IF, IG, IH, II, IK, IL, IM, IN, IO, IP and IQ in which Lₙ is 2-fluoro,4-methoxy, R² is methoxy and R¹ for a compound corresponds in each case to one row of Table A

Table 134

Compounds of the formulae IA, IB, IC, ID, IE, IF, IG, IH, II, IK, IL, IM, IN, IO, IP and IQ in which Lₙ is 2-fluoro,5-methyl, R² is methoxy and R¹ for a compound corresponds in each case to one row of Table A

Table 135

Compounds of the formulae IA, IB, IC, ID, IE, IF, IG, IH, II, IK, IL, IM, IN, IO, IP and IQ in which Lₙ is pentafluoro, R² is methoxy and R¹ for a compound corresponds in each case to one row of Table A

Table 136

Compounds of the formulae IA, IB, IC, ID, IE, IF, IG, IH, II, IK, IL, IM, IN, IO, IP and IQ in which Lₙ is 2-fluoro,6-chloro, R² is cyano and R¹ for a compound corresponds in each case to one row of Table A

Table 137

Compounds of the formulae IA, IB, IC, ID, IE, IF, IG, IH, II, IK, IL, IM, IN, IO, IP and IQ in which Lₙ is 2,6-difluoro, R² is cyano and R¹ for a compound corresponds in each case to one row of Table A

Table 138

Compounds of the formulae IA, IB, IC, ID, IE, IF, IG, IH, II, IK, IL, IM, IN, IO, IP and IQ in which Lₙ is 2,6-dichloro, R² is cyano and R¹ for a compound corresponds in each case to one row of Table A

Table 139

Compounds of the formulae IA, IB, IC, ID, IE, IF, IG, IH, II, IK, IL, IM, IN, IO, IP and IQ in which Lₙ is 2-fluoro,6-methyl, R² is cyano and R¹ for a compound corresponds in each case to one row of Table A

Table 140

Compounds of the formulae IA, IB, IC, ID, IE, IF, IG, IH, II, IK, IL, IM, IN, IO, IP and IQ in which Lₙ is 2,4,6-trifluoro, R² is cyano and R¹ for a compound corresponds in each case to one row of Table A

Table 141

Compounds of the formulae IA, IB, IC, ID, IE, IF, IG, IH, II, IK, IL, IM, IN, IO, IP and IQ in which Lₙ is 2-methyl,4-fluoro, R² is cyano and R¹ for a compound corresponds in each case to one row of Table A

Table 142

Compounds of the formulae IA, IB, IC, ID, IE, IF, IG, IH, II, IK, IL, IM, IN, IO, IP and IQ in which Lₙ is 2-fluoro,4-methoxycarbonyl, R² is cyano and R¹ for a compound corresponds in each case to one row of Table A

Table 143

Compounds of the formulae IA, IB, IC, ID, IE, IF, IG, IH, II, IK, IL, IM, IN, IO, IP and IQ in which Lₙ is 2-fluoro,4-CN, R² is cyano and R¹ for a compound corresponds in each case to one row of Table A

Table 144

Compounds of the formulae IA, IB, IC, ID, IE, IF, IG, IH, II, IK, IL, IM, IN; 10, IP and IQ in which Lₙ is 2,4,5-trifluoro, R² is cyano and R¹ for a compound corresponds in each case to one row of Table A

Table 145

Compounds of the formulae IA, IB, IC, ID, IE, IF, IG, IH, II, IK, IL, IM, IN, IO, IP and IQ in which Lₙ is 2,4-dichloro, R² is cyano and R¹ for a compound corresponds in each case to one row of Table A

Table 146

Compounds of the formulae IA, IB, IC, ID, IE, IF, IG, IH, II, IK, IL, IM, IN, IO, IP and IQ in which Lₙ is 2-chloro, R² is cyano and R¹ for a compound corresponds in each case to one row of Table A

Table 147

Compounds of the formulae IA, IB, IC, ID, IE, IF, IG, IH, II, IK, IL, IM, IN, IO, IP and IQ in which Lₙ is 2-fluoro, R² is cyano and R¹ for a compound corresponds in each case to one row of Table A

Table 148

Compounds of the formulae IA, IB, IC, ID, IE, IF, IG, IH, II, IK, IL, IM, IN, IO, IP and IQ in which Lₙ is 2,4-difluoro, R² is cyano and R¹ for a compound corresponds in each case to one row of Table A

Table 149

Compounds of the formulae IA, IB, IC, ID, IE, IF, IG, IH, II, IK, IL, IM, IN, IO, IP and IQ in which Lₙ is 2-fluoro-4-chloro, R² is cyano and R¹ for a compound corresponds in each case to one row of Table A

Table 150

Compounds of the formulae IA, IB, IC, ID, IE, IF, IG, IH, II, IK, IL, IM, IN, IO, IP and IQ in which Lₙ is 2-chloro-4-fluoro, R² is cyano and R¹ for a compound corresponds in each case to one row of Table A

Table 151

Compounds of the formulae IA, IB, IC, ID, IE, IF, IG, IH, II, IK, IL, IM, IN, IO, IP and IQ in which Lₙ is 2,3-difluoro, R² is cyano and R¹ for a compound corresponds in each case to one row of Table A

Table 152

Compounds of the formulae IA, IB, IC, ID, IE, IF, IG, IH, II, IK, IL, IM, IN, IO, IP and IQ in which Lₙ is 2,5-difluoro, R² is cyano and R¹ for a compound corresponds in each case to one row of Table A

Table 153

Compounds of the formulae IA, IB, IC, ID, IE, IF, IG, IH, II, IK, IL, IM, IN, IO, IP and IQ in which Lₙ is 2,3,4-trifluoro, R² is cyano and R¹ for a compound corresponds in each case to one row of Table A

Table 154

Compounds of the formulae IA, IB, IC, ID, IE, IF, IG, IH, II, IK, IL, IM, IN, IO, IP and IQ in which Lₙ is 2-methyl, R² is cyano and R¹ for a compound corresponds in each case to one row of Table A

Table 155

Compounds of the formulae IA, IB, IC, ID, IE, IF, IG, IH, II, IK, IL, IM, IN, IO, IP and IQ in which Lₙ is 2,4-dimethyl, R² is cyano and R¹ for a compound corresponds in each case to one row of Table A

Table 156

Compounds of the formulae IA, IB, IC, ID, IE, IF, IG, IH, II, IK, IL, IM, IN, IO, IP and IQ in which Lₙ is 2-methyl-4-chloro, R² is cyano and R¹ for a compound corresponds in each case to one row of Table A

Table 157

Compounds of the formulae IA, IB, IC, ID, IE, IF, IG, IH, II, IK, IL, IM, IN, IO, IP and IQ in which Lₙ is 2-fluoro-4-methyl, R² is cyano and R¹ for a compound corresponds in each case to one row of Table A

Table 158

Compounds of the formulae IA, IB, IC, ID, IE, IF, IG, IH, II, IK, IL, IM, IN, IO, IP and IQ in which Lₙ is 2,6-dimethyl, R² is cyano and R¹ for a compound corresponds in each case to one row of Table A

Table 159

Compounds of the formulae IA, IB, IC, ID, IE, IF, IG, IH, II, IK, IL, IM, IN, IO, IP and IQ in which Lₙ is 2,4,6-trimethyl, R² is cyano and R¹ for a compound corresponds in each case to one row of Table A

Table 160

Compounds of the formulae IA, IB, IC, ID, IE, IF, IG, IH, II, IK, IL, IM, IN, IO, IP and IQ in which Lₙ is 2,6-difluoro-4-cyano, R² is cyano and R¹ for a compound corresponds in each case to one row of Table A

Table 161

Compounds of the formulae IA, IB, IC, ID, IE, IF, IG, IH, II, IK, IL, IM, IN, IO, IP and IQ in which Lₙ is 2,6-difluoro-4-methyl, R² is cyano and R¹ for a compound corresponds in each case to one row of Table A

Table 162

Compounds of the formulae IA, IB, IC, ID, IE, IF, IG, IH, II, IK, IL, IM, IN, IO, IP and IQ in which Lₙ is 2,6-difluoro-4-methoxycarbonyl, R² is cyano and R¹ for a compound corresponds in each case to one row of Table A

Table 163

Compounds of the formulae IA, IB, IC, ID, IE, IF, IG, IH, II, IK, IL, IM, IN, IO, IP and IQ in which Lₙ is 2-chloro,4-methoxy, R² is cyano and R¹ for a compound corresponds in each case to one row of Table A

Table 164

Compounds of the formulae IA, IB, IC, ID, IE, IF, IG, IH, II, IK, IL, IM, IN, IO, IP and IQ in which Lₙ is 2-chloro, 4-methyl, R² is cyano and R¹ for a compound corresponds in each case to one row of Table A

Table 165

Compounds of the formulae IA, IB, IC, ID, IE, IF, IG, IH, II, IK, IL, IM, IN, IO, IP and IQ in which Lₙ is 2-chloro,4-methoxycarbonyl, R² is cyano and R¹ for a compound corresponds in each case to one row of Table A

Table 166

Compounds of the formulae IA, IB, IC, ID, IE, IF, IG, IH, II, IK, IL, IM, IN, IO, IP and IQ in which Lₙ is 2-chloro,4-bromo, R² is cyano and R¹ for a compound corresponds in each case to one row of Table A

Table 167

Compounds of the formulae IA, IB, IC, ID, IE, IF, IG, IH, II, IK, IL, IM, IN, IO, IP and IQ in which Lₙ is 2-chloro,4-cyano, R² is cyano and R¹ for a compound corresponds in each case to one row of Table A

Table 168

Compounds of the formulae IA, IB, IC, ID, IE, IF, IG, IH, II, IK, IL, IM, IN, IO, IP and IQ in which Lₙ is 2,6-difluoro,4-methoxy, R² is cyano and R¹ for a compound corresponds in each case to one row of Table A

Table 169

Compounds of the formulae IA, IB, IC, ID, IE, IF, IG, IH, II, IK, IL, IM, IN, IO, IP and IQ in which Lₙ is 2-fluoro,3-methyl, R² is cyano and R¹ for a compound corresponds in each case to one row of Table A

Table 170

Compounds of the formulae IA, IB, IC, ID, IE, IF, IG, IH, II, IK, IL, IM, IN, IO, IP and IQ in which Lₙ is 2,5-dimethyl, R² is cyano and R¹ for a compound corresponds in each case to one row of Table A

Table 171

Compounds of the formulae IA, IB, IC, ID, IE, IF, IG, IH, II, IK, IL, IM, IN, IO, IP and IQ in which Lₙ is 2-methyl,4-cyano, R² is cyano and R¹ for a compound corresponds in each case to one row of Table A

Table 172

Compounds of the formulae IA, IB, IC, ID, IE, IF, IG, IH, II, IK, IL, IM, IN, IO, IP and IQ in which Lₙ is 2-methyl,4-bromo, R² is cyano and R¹ for a compound corresponds in each case to one row of Table A

Table 173

Compounds of the formulae IA, IB, IC, ID, IE, IF, IG, IH, II, IK, IL, IM, IN, IO, IP and IQ in which Lₙ is 2-methyl,4-fluoro, R² is cyano and R¹ for a compound corresponds in each case to one row of Table A

Table 174

Compounds of the formulae IA, IB, IC, ID, IE, IF, IG, IH, II, IK, IL, IM, IN, IO, IP and IQ in which Lₙ is 2-methyl,4-methoxy, R² is cyano and R¹ for a compound corresponds in each case to one row of Table A

Table 175

Compounds of the formulae IA, IB, IC, ID, IE, IF, IG, IH, II, IK, IL, IM, IN, IO, IP and IQ in which Lₙ is 2-methyl,4-methoxycarbonyl, R² is cyano and R¹ for a compound corresponds in each case to one row of Table A

Table 176

Compounds of the formulae IA, IB, IC, ID, IE, IF, IG, IH, II, IK, IL, IM, IN, IO, IP and IQ in which Lₙ is 2,5-dimethyl,4-bromo, R² is cyano and R¹ for a compound corresponds in each case to one row of Table A

Table 177

Compounds of the formulae IA, IB, IC, ID, IE, IF, IG, IH, II, IK, IL, IM, IN, IO, IP and IQ in which Lₙ is 2-fluoro,4-bromo, R² is cyano and R¹ for a compound corresponds in each case to one row of Table A

Table 178

Compounds of the formulae IA, IB, IC, ID, IE, IF, IG, IH, II, IK, IL, IM, IN, IO, IP and IQ in which Lₙ is 2-fluoro,4-methoxy, R² is cyano and R¹ for a compound corresponds in each case to one row of Table A

Table 179

Compounds of the formulae IA, IB, IC, ID, IE, IF, IG, IH, II, IK, IL, IM, IN, IO, IP and IQ in which Lₙ is 2-fluoro,5-methyl, R² is cyano and R¹ for a compound corresponds in each case to one row of Table A

Table 180

Compounds of the formulae IA, IB, IC, ID, IE, IF, IG, IH, II, IK, IL, IM, IN, IO, IP and IQ in which Lₙ is pentafluoro, R² is cyano

TABLE A

| No. | R¹ |
|---|---|
| A-1 | CH₃ |
| A-2 | CH₂CH₃ |
| A-3 | CH₂CH₂CH₃ |
| A-4 | CH(CH₃)₂ |
| A-5 | CH₂CH(CH₃)₂ |
| A-6 | (±)CH(CH₃)CH₂CH₃ |
| A-7 | (R)CH(CH₃)CH₂CH₃ |
| A-8 | (S)CH(CH₃)CH₂CH₃ |
| A-9 | (CH₂)₃CH₃ |
| A-10 | C(CH₃)₃ |
| A-11 | (CH₂)₄CH₃ |
| A-12 | CH(CH₂CH₃)₂ |
| A-13 | CH₂CH₂CH(CH₃)₂ |
| A-14 | (±)CH(CH₃)(CH₂)₂CH₃ |
| A-15 | (R)CH(CH₃)(CH₂)₂CH₃ |
| A-16 | (S)CH(CH₃)(CH₂)₂CH₃ |
| A-17 | (±)CH₂CH(CH₃)CH₂CH₃ |
| A-18 | (R)CH₂CH(CH₃)CH₂CH₃ |
| A-19 | (S)CH₂CH(CH₃)CH₂CH₃ |
| A-20 | (±)CH(CH₃)CH(CH₃)₂ |
| A-21 | (R)CH(CH₃)CH(CH₃)₂ |
| A-22 | (S)CH(CH₃)CH(CH₃)₂ |
| A-23 | (CH₂)₅CH₃ |
| A-24 | (±,±)CH(CH₃)CH(CH₃)CH₂CH₃ |
| A-25 | (±,R)CH(CH₃)CH(CH₃)CH₂CH₃ |
| A-26 | (±,S)CH(CH₃)CH(CH₃)CH₂CH₃ |
| A-27 | (±)CH₂CH(CH₃)CF₃ |
| A-28 | (R)CH₂CH(CH₃)CF₃ |
| A-29 | (S)CH₂CH(CH₃)CF₃ |
| A-30 | (±)CH₂CH(CF₃)CH₂CH₃ |
| A-31 | (R)CH₂CH(CF₃)CH₂CH₃ |
| A-32 | (S)CH₂CH(CF₃)CH₂CH₃ |
| A-33 | (±,±)CH(CH₃)CH(CH₃)CF₃ |
| A-34 | (±,R)CH(CH₃)CH(CH₃)CF₃ |
| A-35 | (±,S)CH(CH₃)CH(CH₃)CF₃ |
| A-36 | (±,±)CH(CH₃)CH(CF₃)CH₂CH₃ |
| A-37 | (±,R)CH(CH₃)CH(CF₃)CH₂CH₃ |
| A-38 | (±,S)CH(CH₃)CH(CF₃)CH₂CH₃ |
| A-39 | CF₃ |
| A-40 | CF₂CF₃ |
| A-41 | CF₂CF₂CF₃ |
| A-42 | c-C₃H₅ |
| A-43 | (1-CH₃)-c-C₃H₄ |
| A-44 | c-C₅H₉ |
| A-45 | c-C₆H₁₁ |
| A-46 | (4-CH₃)-c-C₆H₁₀ |
| A-47 | CH₂C(CH₃)═CH₂ |
| A-48 | CH₂CH₂C(CH₃)═CH₂ |
| A-49 | CH₂—C(CH₃)₃ |
| A-50 | CH₂—Si(CH₃)₃ |
| A-51 | n-C₆H₁₃ |
| A-52 | (CH₂)₃—CH(CH₃)₂ |
| A-53 | (CH₂)₂—CH(CH₃)—C₂H₅ |
| A-54 | CH₂—CH(CH₃)-n-C₃H₇ |
| A-55 | CH(CH₃)-n-C₄H₉ |
| A-56 | CH₂—CH(C₂H₅)₂ |
| A-57 | CH(C₂H₅)-n-C₃H₇ |
| A-58 | CH₂-c-C₅H₉ |
| A-59 | CH₂—CH(CH₃)—CH(CH₃)₂ |
| A-60 | CH(CH₃)—CH₂CH(CH₃)₂ |
| A-61 | CH(CH₃)—CH(CH₃)—C₂H₅ |
| A-62 | CH(CH₃)—C(CH₃)₃ |
| A-63 | (CH₂)₂—C(CH₃)₃ |
| A-64 | CH₂—C(CH₃)₂—C₂H₅ |
| A-65 | 2-CH₃-c-C₅H₈ |
| A-66 | 3-CH₃-c-C₅H₈ |
| A-67 | C(CH₃)₂-n-C₃H₇ |
| A-68 | (CH₂)₆—CH₃ |
| A-69 | (CH₂)₄—CH(CH₃)₂ |
| A-70 | (CH₂)₃—CH(CH₃)—C₂H₅ |
| A-71 | (CH₂)₂—CH(CH₃)-n-C₃H₇ |
| A-72 | CH₂—CH(CH₃)-n-C₄H₉ |
| A-73 | CH(CH₃)-n-C₅H₁₁ |
| A-74 | (CH₂)₃C(CH₃)₃ |
| A-75 | (CH₂)₂CH(CH₃)—CH(CH₃)₂ |
| A-76 | (CH₂)CH(CH₃)—CH₂CH(CH₃)₂ |
| A-77 | CH(CH₃)(CH₂)₂—CH(CH₃)₂ |
| A-78 | (CH₂)₂C(CH₃)₂C₂H₅ |
| A-79 | CH₂CH(CH₃)CH(CH₃)C₂H₅ |
| A-80 | CH(CH₃)CH₂CH(CH₃)C₂H₅ |
| A-81 | CH₂C(CH₃)₂-n-C₃H₇ |
| A-82 | CH(CH₃)CH(CH₃)-n-C₃H₇ |
| A-83 | C(CH₃)₂-n-C₄H₉ |
| A-84 | (CH₂)₂CH(C₂H₅)₂ |
| A-85 | CH₂CH(C₂H₅)-n-C₃H₇ |
| A-86 | CH(C₂H₅)-n-C₄H₉ |
| A-87 | CH₂CH(CH₃)C(CH₃)₃ |
| A-88 | CH(CH₃)CH₂C(CH₃)₃ |
| A-89 | CH₂C(CH₃)₂CH(CH₃)₂ |
| A-90 | CH₂CH(C₂H₅)CH(CH₃)₂ |
| A-91 | CH(CH₃)CH(CH₃)CH(CH₃)₂ |
| A-92 | C(CH₃)₂CH₂CH(CH₃)₂ |
| A-93 | CH(C₂H₅)CH₂CH(CH₃)₂ |
| A-94 | CH(CH₃)C(CH₃)₂C₂H₅ |
| A-95 | CH(CH₃)CH(C₂H₅)₂ |
| A-96 | C(CH₃)₂CH(CH₃)C₂H₅ |
| A-97 | CH(C₂H₅)CH(CH₃)C₂H₅ |
| A-98 | C(CH₃)(C₂H₅)-n-C₃H₇ |
| A-99 | CH(n-C₃H₇)₂ |
| A-100 | CH(n-C₃H₇)CH(CH₃)₂ |
| A-101 | C(CH₃)₂C(CH₃)₃ |
| A-102 | C(CH₃)(C₂H₅)—CH(CH₃)₂ |
| A-103 | C(C₂H₅)₃ |
| A-104 | (3-CH₃)-c-C₆H₁₀ |
| A-105 | (2-CH₃)-c-C₆H₁₀ |
| A-106 | n-C₈H₁₇ |
| A-107 | CH₂C(═NO—CH₃)CH₃ |
| A-108 | CH₂C(═NO—C₂H₅)CH₃ |
| A-109 | CH₂C(═NO-n-C₃H₇)CH₃ |
| A-110 | CH₂C(═NO-i-C₃H₇)CH₃ |
| A-111 | CH(CH₃)C(═NOCH₃)CH₃ |
| A-112 | CH(CH₃)C(═NOC₂H₅)CH₃ |
| A-113 | CH(CH₃)C(═NO-n-C₃H₇)CH₃ |
| A-114 | CH(CH₃)C(═NO-i-C₃H₇)CH₃ |
| A-115 | C(═NOCH₃)C(═NOCH₃)CH₃ |
| A-116 | C(═NOCH₃)C(═NOC₂H₅)CH₃ |
| A-117 | C(═NOCH₃)C(═NO-n-C₃H₇)CH₃ |
| A-118 | C(═NOCH₃)C(═NO-i-C₃H₇)CH₃ |
| A-119 | C(═NOC₂H₅)C(═NOCH₃)CH₃ |
| A-120 | C(═NOC₂H₅)C(═NOC₂H₅)CH₃ |
| A-121 | C(═NOC₂H₅)C(═NO-n-C₃H₇)CH₃ |
| A-122 | C(═NOC₂H₅)C(═NO-i-C₃H₇)CH₃ |
| A-123 | CH₂C(═NO—CH₃)C₂H₅ |
| A-124 | CH₂C(═NO—C₂H₅)C₂H₅ |
| A-125 | CH₂C(═NO-n-C₃H₇)C₂H₅ |
| A-126 | CH₂C(═NO-i-C₃H₇)C₂H₅ |
| A-127 | CH(CH₃)C(═NOCH₃)C₂H₅ |
| A-128 | CH(CH₃)C(═NOC₂H₅)C₂H₅ |
| A-129 | CH(CH₃)C(═NO-n-C₃H₇)C₂H₅ |
| A-130 | CH(CH₃)C(═NO-n-C₃H₇)C₂H₅ |
| A-131 | C(═NOCH₃)C(═NOCH₃)C₂H₅ |
| A-132 | C(═NOCH₃)C(═NOC₂H₅)C₂H₅ |
| A-133 | C(═NOCH₃)C(═NO-n-C₃H₇)C₂H₅ |
| A-134 | C(═NOCH₃)C(═NO-i-C₃H₇)C₂H₅ |
| A-135 | C(═NOC₂H₅)C(═NOCH₃)C₂H₅ |
| A-136 | C(═NOC₂H₅)C(═NOC₂H₅)C₂H₅ |
| A-137 | C(═NOC₂H₅)C(═NO-n-C₃H₇)C₂H₅ |
| A-138 | C(═NOC₂H₅)C(═NO-i-C₃H₇)C₂H₅ |
| A-139 | CH═CH—CH₂CH₃ |
| A-140 | CH₂—CH═CH—CH₃ |
| A-141 | CH₂—CH₂—CH═CH₂ |
| A-142 | C(CH₃)₂CH₂CH₃ |
| A-143 | CH═C(CH₃)₂ |
| A-144 | C(═CH₂)—CH₂CH₃ |
| A-145 | C(CH₃)═CH—CH₃ |
| A-146 | CH(CH₃)CH═CH₂ |
| A-147 | CH═CH-n-C₃H₇ |
| A-148 | CH₂—CH═CH—C₂H₅ |
| A-149 | (CH₂)₂—CH═CH—CH₃ |
| A-150 | (CH₂)₃—CH═CH₂ |
| A-151 | CH═CH—CH(CH₃)₂ |
| A-152 | CH₂—CH═C(CH₃)₂ |
| A-153 | (CH₂)₂—C(CH₃)═CH₂ |
| A-154 | CH═C(CH₃)—C₂H₅ |
| A-155 | CH₂—C(═CH₂)—C₂H₅ |
| A-156 | CH₂—C(CH₃)═CH—CH₃ |
| A-157 | CH₂—CH(CH₃)—CH═CH₂ |
| A-158 | C(═CH₂)—CH₂—CH₂—CH₃ |
| A-159 | C(CH₃)═CH—CH₂—CH₃ |
| A-160 | CH(CH₃)—CH═CH—CH₃ |
| A-161 | CH(CH₃)—CH₂—CH═CH₂ |
| A-162 | C(═CH₂)CH(CH₃)₂ |
| A-163 | C(CH₃)═C(CH₃)₂ |
| A-164 | CH(CH₃)—C(═CH₂)—CH₃ |
| A-165 | C(CH₃)₂—CH═CH₂ |
| A-166 | C(C₂H₅)═CH—CH₃ |
| A-167 | CH(C₂H₅)—CH═CH₂ |
| A-168 | CH═CH—CH₂—CH₂—CH₂—CH₃ |
| A-169 | CH₂—CH═CH—CH₂—CH₂—CH₃ |
| A-170 | CH₂—CH₂—CH═CH—CH₂—CH₃ |
| A-171 | CH₂—CH₂—CH₂—CH═CH—CH₃ |
| A-172 | CH₂—CH₂—CH₂—CH₂—CH═CH₂ |
| A-173 | CH═CH—CH₂—CH(CH₃)CH₃ |
| A-174 | CH₂—CH═CH—CH(CH₃)CH₃ |
| A-175 | CH₂—CH₂—CH═C(CH₃)CH₃ |
| A-176 | CH₂—CH₂—CH₂—C(CH₃)═CH₂ |
| A-177 | CH═CH—CH(CH₃)—CH₂—CH₃ |
| A-178 | CH₂—CH═C(CH₃)—CH₂—CH₃ |
| A-179 | CH₂—CH₂—C(═CH₂)—CH₂—CH₃ |
| A-180 | CH₂—CH₂—C(CH₃)═CH—CH₃ |
| A-181 | CH₂—CH₂—CH(CH₃)—CH═CH₂ |
| A-182 | CH═C(CH₃)—CH₂—CH₂—CH₃ |
| A-183 | CH₂—C(═CH₂)—CH₂—CH₂—CH₃ |
| A-184 | CH₂—C(CH₃)═CH—CH₂—CH₃ |
| A-185 | CH₂—CH(CH₃)—CH═CH—CH₃ |
| A-186 | CH₂—CH(CH₃)—CH₂—CH═CH₂ |
| A-187 | C(═CH₂)—CH₂—CH₂—CH₂—CH₃ |
| A-188 | C(CH₃)═CH—CH₂—CH₂—CH₃ |
| A-189 | CH(CH₃)—CH═CH—CH₂—CH₃ |
| A-190 | CH(CH₃)—CH₂—CH═CH—CH₃ |
| A-191 | CH(CH₃)—CH₂—CH₂—CH═CH₂ |
| A-192 | CH═CH—C(CH₃)₃ |
| A-193 | CH═C(CH₃)—CH(CH₃)—CH₃ |
| A-194 | CH₂—C(═CH₂)—CH(CH₃)—CH₃ |
| A-195 | CH₂—C(CH₃)═C(CH₃)—CH₃ |
| A-196 | CH₂—CH(CH₃)—C(═CH₂)—CH₃ |
| A-197 | C(═CH₂)—CH₂—CH(CH₃)—CH₃ |
| A-198 | C(CH₃)═CH—CH(CH₃)—CH₃ |
| A-199 | CH(CH₃)—CH═C(CH₃)—CH₃ |
| A-200 | CH(CH₃)—CH₂—C(═CH₂)—CH₃ |
| A-201 | CH═C(CH₂—CH₃)—CH₂—CH₃ |
| A-202 | CH₂—C(═CH—CH₃)—CH₂—CH₃ |
| A-203 | CH₂—CH(CH═CH₂)—CH₂—CH₃ |
| A-204 | C(═CH—CH₃)—CH₂—CH₂—CH₃ |
| A-205 | CH(CH═CH₂)—CH₂—CH₂—CH₃ |
| A-206 | C(CH₂—CH₃)═CH—CH₂—CH₃ |
| A-207 | CH(CH₂—CH₃)—CH═CH—CH₃ |
| A-208 | CH(CH₂—CH₃)—CH₂—CH═CH₂ |
| A-209 | CH₂—C(CH₃)₂—CH═CH₂ |
| A-210 | C(═CH₂)—CH(CH₃)—CH₂—CH₃ |
| A-211 | C(CH₃)═C(CH₃)—CH₂—CH₃ |
| A-212 | CH(CH₃)—C(═CH₂)—CH₂—CH₃ |
| A-213 | CH(CH₃)—C(CH₃)═CH—CH₃ |
| A-214 | CH(CH₃)—CH(CH₃)—CH═CH₂ |
| A-215 | C(CH₃)₂—CH═CH—CH₃ |
| A-216 | C(CH₃)₂—CH₂—CH═CH₂ |
| A-217 | C(═CH₂)—C(CH₃)₃ |
| A-218 | C(═CH—CH₃)—CH(CH₃)—CH₃ |
| A-219 | CH(CH═CH₂)—CH(CH₃)—CH₃ |
| A-220 | C(CH₂—CH₃)═C(CH₃)—CH₃ |
| A-221 | CH(CH₂—CH₃)—C(═CH₂)—CH₃ |
| A-222 | C(CH₃)₂—C(═CH₂)—CH₃ |
| A-223 | C(CH₃)(CH═CH₂)—CH₂—CH₃ |
| A-224 | C(CH₃)(CH₂CH₃)—CH₂—CH₂—CH₃ |
| A-225 | CH(CH₂CH₃)—CH(CH₃)—CH₂—CH₃ |
| A-226 | CH(CH₂CH₃)—CH₂—CH(CH₃)—CH₃ |
| A-227 | C(CH₃)₂—C(CH₃)₃ |
| A-228 | C(CH₂—CH₃)—C(CH₃)₃ |
| A-229 | C(CH₃)(CH₂—CH₃)—CH(CH₃)₂ |
| A-230 | CH(CH(CH₃)₂)—CH(CH₃)₂ |
| A-231 | CH═CH—CH₂—CH₂—CH₂—CH₂—CH₃ |
| A-232 | CH₂—CH═CH—CH₂—CH₂—CH₂—CH₃ |
| A-233 | CH₂—CH₂—CH═CH—CH₂—CH₂—CH₃ |
| A-234 | CH₂—CH₂—CH₂—CH═CH—CH₂—CH₃ |
| A-235 | CH₂—CH₂—CH₂—CH₂—CH═CH—CH₃ |
| A-236 | CH₂—CH₂—CH₂—CH₂—CH₂—CH═CH₂ |
| A-237 | CH═CH—CH₂—CH₂—CH(CH₃)—CH₃ |
| A-238 | CH₂—CH═CH—CH₂—CH(CH₃)—CH₃ |
| A-239 | CH₂—CH₂—CH═CH—CH(CH₃)—CH₃ |
| A-240 | CH₂—CH₂—CH₂—CH═C(CH₃)—CH₃ |
| A-241 | CH₂—CH₂—CH₂—CH₂—C(═CH₂)—CH₃ |
| A-242 | CH═CH—CH₂—CH(CH₃)—CH₂—CH₃ |
| A-243 | CH₂—CH═CH—CH(CH₃)—CH₂—CH₃ |
| A-244 | CH₂—CH₂—CH═C(CH₃)—CH₂—CH₃ |
| A-245 | CH₂—CH₂—CH₂—C(═CH₂)—CH₂—CH₃ |
| A-246 | CH₂—CH₂—CH₂—C(CH₃)═CH—CH₃ |
| A-247 | CH₂—CH₂—CH₂—CH(CH₃)—CH═CH₂ |
| A-248 | CH═CH—CH(CH₃)—CH₂—CH₂—CH₃ |
| A-249 | CH₂—CH═C(CH₃)—CH₂—CH₂—CH₃ |
| A-250 | CH₂—CH₂—C(═CH₂)—CH₂—CH₂—CH₃ |
| A-251 | CH₂—CH₂—C(CH₃)═CH—CH₂—CH₃ |
| A-252 | CH₂—CH₂—CH(CH₃)—CH═CH—CH₃ |
| A-253 | CH₂—CH₂—CH(CH₃)—CH₂—CH═CH₂ |
| A-254 | CH═C(CH₃)—CH₂—CH₂—CH₂—CH₃ |
| A-255 | CH₂—C(═CH₂)—CH₂—CH₂—CH₂—CH₃ |
| A-256 | CH₂—C(CH₃)═CH—CH₂—CH₂—CH₃ |
| A-257 | CH₂—CH(CH₃)—CH═CH—CH₂—CH₃ |
| A-258 | CH₂—CH(CH₃)—CH₂—CH═CH—CH₃ |
| A-259 | CH₂—CH(CH₃)—CH₂—CH₂—CH═CH₂ |
| A-260 | C(═CH₂)—CH₂—CH₂—CH₂—CH₂—CH₃ |
| A-261 | C(CH₃)═CH—CH₂—CH₂—CH₂—CH₃ |
| A-262 | CH(CH₃)—CH═CH—CH₂—CH₂—CH₃ |
| A-263 | CH(CH₃)—CH₂—CH═CH—CH₂—CH₃ |
| A-264 | CH(CH₃)—CH₂—CH₂—CH═CH—CH₃ |
| A-265 | CH(CH₃)—CH₂—CH₂—CH₂—CH═CH₂ |
| A-266 | CH═CH—CH₂—C(CH₃)₃ |
| A-267 | CH₂—CH═CH—C(CH₃)₃ |
| A-268 | CH═CH—CH(CH₃)—CH(CH₃)₂ |
| A-269 | CH₂—CH═C(CH₃)—CH(CH₃)₂ |
| A-270 | CH₂—CH₂—C(═CH₂)—CH(CH₃)₂ |
| A-271 | CH₂—CH₂—C(CH₃)═C(CH₃)₂ |
| A-272 | CH₂—CH₂—CH(CH₃)—C(═CH₂)—CH₃ |
| A-273 | CH═C(CH₃)—CH₂—CH(CH₃)₂ |
| A-274 | CH₂—C(═CH₂)—CH₂—CH(CH₃)₂ |
| A-275 | CH₂—C(CH₃)═CH—CH(CH₃)₂ |
| A-276 | CH₂—CH(CH₃)—CH═C(CH₃)₂ |
| A-277 | CH₂—CH(CH₃)—CH₂—C(═CH₂)—CH₃ |
| A-278 | C(═CH₂)—CH₂—CH₂—CH(CH₃)₂ |
| A-279 | C(CH₃)═CH—CH₂—CH(CH₃)₂ |
| A-280 | CH(CH₃)—CH═CH—CH(CH₃)₂ |
| A-281 | CH(CH₃)—CH₂—CH═C(CH₃)₂ |
| A-282 | CH(CH₃)—CH₂—CH₂—C(═CH₂)—CH₃ |
| A-283 | CH═CH—C(CH₃)₂—CH₂—CH₃ |
| A-284 | CH₂—CH₂—C(CH₃)₂—CH═CH₂ |
| A-285 | CH═C(CH₃)—CH(CH₃)—CH₂—CH₃ |
| A-286 | CH₂—C(═CH₂)—CH(CH₃)—CH₂—CH₃ |
| A-287 | CH₂—C(CH₃)═C(CH₃)—CH₂—CH₃ |
| A-288 | CH₂—CH(CH₃)—C(═CH₂)—CH₂—CH₃ |
| A-289 | CH₂—CH(CH₃)—C(CH₃)═CH—CH₃ |
| A-290 | CH₂—CH(CH₃)—CH(CH₃)—CH═CH₂ |
| A-291 | C(═CH₂)—CH₂—CH(CH₃)—CH₂—CH₃ |
| A-292 | C(CH₃)═CH—CH(CH₃)—CH₂—CH₃ |
| A-293 | CH(CH₃)—CH═C(CH₃)—CH₂—CH₃ |
| A-294 | CH(CH₃)—CH₂—C(═CH₂)—CH₂—CH₃ |
| A-295 | CH(CH₃)—CH₂—C(CH₃)═CH—CH₃ |
| A-296 | CH(CH₃)—CH₂—CH(CH₃)—CH═CH₂ |
| A-297 | CH₂—C(CH₃)₂—CH═CH—CH₃ |
| A-298 | CH₂—C(CH₃)₂—CH₂—CH═CH₂ |
| A-299 | C(═CH₂)—CH(CH₃)—CH₂—CH₂—CH₃ |
| A-300 | C(CH₃)═C(CH₃)—CH₂—CH₂—CH₃ |
| A-301 | CH(CH₃)—C(═CH₂)—CH₂—CH₂—CH₃ |
| A-302 | CH(CH₃)—C(CH₃)═CH—CH₂—CH₃ |
| A-303 | CH(CH₃)—CH(CH₃)—CH═CH—CH₃ |
| A-304 | CH(CH₃)—CH(CH₃)—CH₂—CH═CH₂ |
| A-305 | C(CH₃)₂—CH═CH—CH₂—CH₃ |
| A-306 | C(CH₃)₂—CH₂—CH═CH—CH₃ |
| A-307 | C(CH₃)₂—CH₂—CH₂—CH═CH₂ |
| A-308 | CH═CH—CH(CH₂—CH₃)—CH₂—CH₃ |
| A-309 | CH₂—CH═C(CH₂—CH₃)—CH₂—CH₃ |
| A-310 | CH₂—CH₂—C(═CH—CH₃)—CH₂—CH₃ |
| A-311 | CH₂—CH₂—CH(CH═CH₂)—CH₂—CH₃ |
| A-312 | CH═C(CH₂—CH₃)—CH₂—CH₂—CH₃ |
| A-313 | CH₂—C(═CH—CH₃)—CH₂—CH₂—CH₃ |
| A-314 | CH₂—CH(CH═CH₂)—CH₂—CH₂—CH₃ |
| A-315 | CH₂—C(CH₂—CH₃)═CH—CH₂—CH₃ |
| A-316 | CH₂—CH(CH₂—CH₃)—CH═CH—CH₃ |
| A-317 | CH₂—CH(CH₂—CH₃)—CH—CH═CH₂ |
| A-318 | C(═CH—CH₃)—CH₂—CH₂—CH₂—CH₃ |
| A-319 | CH(CH═CH₂)—CH₂—CH₂—CH₂—CH₃ |
| A-320 | C(CH₂—CH₃)═CH—CH₂—CH₂—CH₃ |
| A-321 | CH(CH₂—CH₃)—CH═CH—CH₂—CH₃ |
| A-322 | CH(CH₂—CH₃)—CH₂—CH═CH—CH₃ |
| A-323 | CH(CH₂—CH₃)—CH₂—CH₂—CH═CH₂ |
| A-324 | C(═CH—CH₂—CH₃)—CH₂—CH₂—CH₃ |
| A-325 | C(CH═CH—CH₃)—CH₂—CH₂—CH₃ |
| A-326 | C(CH₂—CH═CH₂)—CH₂—CH₂—CH₃ |
| A-327 | CH═C(CH₃)—C(CH₃)₃ |
| A-328 | CH₂—C(═CH₂)—C(CH₃)₃ |
| A-329 | CH₂—C(CH₃)₂—CH(═CH₂)—CH₃ |
| A-330 | C(═CH₂)—CH(CH₃)—CH(CH₃)—CH₃ |
| A-331 | C(CH₃)═C(CH₃)—CH(CH₃)—CH₃ |
| A-332 | CH(CH₃)—C(═CH₂)—CH(CH₃)—CH₃ |
| A-333 | CH(CH₃)—C(CH₃)═C(CH₃)—CH₃ |
| A-334 | CH(CH₃)—CH(CH₃)—C(═CH₂)—CH₃ |
| A-335 | C(CH₃)₂—CH═C(CH₃)—CH₃ |
| A-336 | C(CH₃)₂—CH₂—C(═CH₂)—CH₃ |
| A-337 | C(CH₃)₂—C(═CH₂)—CH₂—CH₃ |
| A-338 | C(CH₃)₂—C(CH₃)═CH—CH₃ |
| A-339 | C(CH₃)₂—CH(CH₃)CH═CH₂ |
| A-340 | CH(CH₂—CH₃)—CH₂—CH(CH₃)—CH₃ |
| A-341 | CH(CH₂—CH₃)—CH(CH₃)—CH₂—CH₃ |
| A-342 | C(CH₃)(CH₂—CH₃)—CH₂—CH₂—CH₃ |
| A-343 | CH(i-C₃H₇)—CH₂—CH₂—CH₃ |
| A-344 | CH═C(CH₂—CH₃)—CH(CH₃)—CH₃ |
| A-345 | CH₂—C(═CH—CH₃)—CH(CH₃)—CH₃ |
| A-346 | CH₂—CH(CH═CH₂)—CH(CH₃)—CH₃ |
| A-347 | CH₂—C(CH₂—CH₃)═C(CH₃)—CH₃ |
| A-348 | CH₂—CH(CH₂—CH₃)—C(═CH₂)—CH₃ |
| A-349 | CH₂—C(CH₃)(CH═CH₂)—CH₂—CH₃ |
| A-350 | C(═CH₂)—CH(CH₂—CH₃)—CH₂—CH₃ |
| A-351 | C(CH₃)═C(CH₂—CH₃)—CH₂—CH₃ |
| A-352 | CH(CH₃)—C(═CH—CH₃)—CH₂—CH₃ |
| A-353 | CH(CH₃)—CH(CH═CH₂)—CH₂—CH₃ |
| A-354 | CH═C(CH₂—CH₃)—CH(CH₃)—CH₃ |
| A-355 | CH₂—C(═CH—CH₃)—CH(CH₃)—CH₃ |
| A-356 | CH₂—CH(CH═CH₂)—CH(CH₃)—CH₃ |
| A-357 | CH₂—C(CH₂—CH₃)═C(CH₃)—CH₃ |
| A-358 | CH₂—CH(CH₂—CH₃)—C(═CH₂)—CH₃ |
| A-359 | C(═CH—CH₃)—CH₂—CH(CH₃)—CH₃ |
| A-360 | CH(CH═CH₂)—CH₂—CH(CH₃)—CH₃ |
| A-361 | C(CH₂—CH₃)═CH—CH(CH₃)—CH₃ |
| A-362 | CH(CH₂—CH₃)CH═C(CH₃)—CH₃ |
| A-363 | CH(CH₂—CH₃)CH₂—C(═CH₂)—CH₃ |
| A-364 | C(═CH—CH₃)CH(CH₃)—CH₂—CH₃ |
| A-365 | CH(CH═CH₂)CH(CH₃)—CH₂—CH₃ |
| A-366 | C(CH₂—CH₃)═C(CH₃)—CH₂—CH₃ |
| A-367 | CH(CH₂—CH₃)—C(═CH₂)—CH₂—CH₃ |
| A-368 | CH(CH₂—CH₃)—C(CH₃)═CH—CH₃ |
| A-369 | CH(CH₂—CH₃)—CH(CH₃)—CH═CH₂ |
| A-370 | C(CH₃)(CH═CH₂)—CH₂—CH₂—CH₃ |
| A-371 | C(CH₃)(CH₂—CH₃)—CH═CH—CH₃ |
| A-372 | C(CH₃)(CH₂—CH₃)—CH₂—CH═CH₂ |
| A-373 | C[═C(CH₃)—CH₃]—CH₂—CH₂—CH₃ |
| A-374 | CH[C(═CH₂)—CH₃]—CH₂—CH₂—CH₃ |
| A-375 | C(i-C₃H₇)═CH—CH₂—CH₃ |
| A-376 | CH(i-C₃H₇)—CH═CH—CH₃ |
| A-377 | CH(i-C₃H₇)—CH₂—CH═CH₂ |
| A-378 | C(═CH—CH₃)—C(CH₃)₃ |
| A-379 | CH(CH═CH₂)—C(CH₃)₃ |
| A-380 | C(CH₃)(CH═CH₂)CH(CH₃)—CH₃ |
| A-381 | C(CH₃)(CH₂—CH₃)C(═CH₂)—CH₃ |
| A-382 | 2-CH₃-cyclohex-1-enyl |
| A-383 | [2-(═CH₂)]-c-C₆H₉ |
| A-384 | 2-CH₃-cyclohex-2-enyl |
| A-385 | 2-CH₃-cyclohex-3-enyl |
| A-386 | 2-CH₃-cyclohex-4-enyl |
| A-387 | 2-CH₃-cyclohex-5-enyl |
| A-388 | 2-CH₃-cyclohex-6-enyl |
| A-389 | 3-CH₃-cyclohex-1-enyl |
| A-390 | 3-CH₃-cyclohex-2-enyl |
| A-391 | [3-(═CH₂)]-c-C₆H₉ |
| A-392 | 3-CH₃-cyclohex-3-enyl |
| A-393 | 3-CH₃-cyclohex-4-enyl |
| A-394 | 3-CH₃-cyclohex-5-enyl |
| A-395 | 3-CH₃-cyclohex-6-enyl |
| A-396 | 4-CH₃-cyclohex-1-enyl |
| A-397 | 4-CH₃-cyclohex-2-enyl |
| A-398 | 4-CH₃-cyclohex-3-enyl |
| A-399 | [4-(═CH₂)]-c-C₆H₉ |

The compounds I are suitable as fungicides. They are distinguished by an outstanding effectiveness against a broad spectrum of phytopathogenic fungi, especially from the classes of the Ascomycetes, Deuteromycetes, Oomycetes and Basidiomycetes. Some are systemically effective and they can be used in plant protection as foliar and soil fungicides.

They are particularly important in the control of a multitude of fungi on various cultivated plants, such as wheat, rye, barley, oats, rice, corn, grass, bananas, cotton, soybean, coffee, sugar cane, vines, fruits and ornamental plants, and vegetables, such as cucumbers, beans, tomatoes, potatoes and cucurbits, and on the seeds of these plants.

They are especially suitable for controlling the following plant diseases:
Alternaria species on fruit and vegetables,
Bipolaris and Drechslera species on cereals, rice and lawns,
Blumeria graminis (powdery mildew) on cereals,
Botrytis cinerea (gray mold) on strawberries, vegetables, ornamental plants and grapevines,
Erysiphe cichoracearum and Sphaerotheca fuliginea on cucurbits,
Fusarium and Verticillium species on various plants,
Mycosphaerella species on cereals, bananas and peanuts,
Phytophthora infestans on potatoes and tomatoes,
Plasmopara viticola on grapevines,
Podosphaera leucotricha on apples,
Pseudocercosporella herpotrichoides on wheat and barley,
Pseudoperonospora species on hops and cucumbers,
Puccinia species on cereals,
Pyricularia oryzae on rice,
Rhizoctonia species on cotton, rice and lawns,
Septoria tritici and Stagonospora nodorum on wheat,
Uncinula necator on grapevines,
Ustilago species on cereals and sugar cane, and
Venturia species (scab) on apples and pears.

The compounds I are also suitable for controlling harmful fungi, such as Paecilomyces variotii, in the protection of materials (e.g. wood, paper, paint dispersions, fibers or fabrics) and in the protection of stored products.

The compounds I are employed by treating the fungi or the plants, seeds, materials or soil to be protected from fungal attack with a fungicidally effective amount of the active compounds. The application can be carried out both before and after the infection of the materials, plants or seeds by the fungi.

The fungicidal compositions generally comprise between 0.1 and 95%, preferably between 0.5 and 90%, by weight of active compound.

When employed in plant protection, the amounts applied are, depending on the kind of effect desired, between 0.01 and 2.0 kg of active compound per ha.

In seed treatment, amounts of active compound of 0.001 to 0.1 g, preferably 0.01 to 0.05 g, per kilogram of seed are generally necessary.

When used in the protection of materials or stored products, the amount of active compound applied depends on the kind of application area and on the effect desired. Amounts customarily applied in the protection of materials are, for example, 0.001 g to 2 kg, preferably 0.005 g to 1 kg, of active compound per cubic meter of treated material.

The compounds I can be converted to the usual formulations, e.g. solutions, emulsions, suspensions, dusts, powders, pastes and granules. The application form depends on the respective use intended; it should in any case guarantee a fine and uniform distribution of the compound according to the invention.

The formulations are prepared in a known manner, for example by extending the active compound with solvents and/or carriers, if desired using emulsifiers and dispersants. Solvents/auxiliaries suitable for this purpose are essentially:
water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (NMP, NOP), acetates (glycol diacetate), glycols, fatty acid dimethylamides, fatty acids and fatty acid esters. In principle, solvent mixtures may also be used.
carriers such as ground natural minerals (for example kaolins, clays, talc, chalk) and ground synthetic minerals (for example highly disperse silica, silicates); emulsifiers such as nonionogenic and anionic emulsifiers (for example polyoxyethylene fatty alcohol ethers, alkylsulfonates and arylsulfonates) and dispersants such as lignosulfite waste liquors and methylcellulose.

Suitable surfactants used are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenyl ether, ethoxylated isooctylphenol, octylphenol, nonylphenol, alkylphenyl polyglycol ethers, tributylphenyl polyglycol ether, tristearylphenyl polyglycol ether, alkylaryl polyether alcohols, alcohol and fatty alcohol ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Substances which are suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, highly polar solvents, for example dimethyl sulfoxide, N-methylpyrrolidone and water.

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active compounds. The active compounds are employed in a purity of from 90% to 100%, preferably 95% to 100% (according to NMR spectrum).

The following are examples of formulations: 1. Products for dilution with water

A) Water-Soluble Concentrates (SL)

10 parts by weight of the active compounds are dissolved in water or in a water-soluble solvent. As an alternative, wetters or other auxiliaries are added. The active compound dissolves upon dilution with water.

B) Dispersible Concentrates (DC)

20 parts by weight of the active compounds are dissolved in cyclohexanone with addition of a dispersant, for example polyvinylpyrrolidone. Dilution with water gives a dispersion.

C) Emulsifiable Concentrates (EC)

15 parts by weight of the active compounds are dissolved in xylene with addition of calcium dodecylbenzenesulfonate and castor oil ethoxylate (in each case 5% strength). Dilution with water gives an emulsion.

D) Emulsions (EW, EO)

40 parts by weight of the active compounds are dissolved in xylene with addition of calcium dodecylbenzenesulfonate and castor oil ethoxylate (in each case 5% strength). This mixture is introduced into water by means of an emulsifying machine (Ultraturrax) and made into a homogeneous emulsion. Dilution with water gives an emulsion.

E) Suspensions (SC, OD)

In an agitated ball mill, 20 parts by weight of the active compounds are comminuted with addition of dispersants, wetters and water or an organic solvent to give a fine active compound suspension. Dilution with water gives a stable suspension of the active compound.

F) Water-Dispersible Granules and Water-Soluble Granules (WG, SG)

50 parts by weight of the active compounds are ground finely with addition of dispersants and wetters and prepared as water-dispersible or water-soluble granules by means of technical appliances (for example extrusion, spray tower, fluidized bed). Dilution with water gives a stable dispersion or solution of the active compound.

G) Water-Dispersible Powders and Water-Soluble Powders (WP, SP)

75 parts by weight of the active compounds are ground in a rotor-stator mill with addition of dispersants, wetters and silica gel. Dilution with water gives a stable dispersion or solution of the active compound.

2. Products to be Applied Undiluted

30H) Dustable powders (DP) parts by weight of the active compounds are ground finely and mixed intimately with 95% of finely divided kaolin. This gives a dustable product.

I) Granules (GR, FG, GG, MG)

0.5 part by weight of the active compounds is ground finely and associated with 95.5% carriers. Current methods are extrusion, spray-drying or the fluidized bed. This gives granules to be applied undiluted.

J) ULV Solutions (UL)

10 parts by weight of the active compounds are dissolved in an organic solvent, for example xylene. This gives a product to be applied undiluted.

The active compounds can be used as such, in the form of their formulations- or of the application forms prepared therefrom, e.g. in the form of directly sprayable solutions, powders, suspensions or dispersions, emulsions, oil dispersions, pastes, dusts, compositions for broadcasting or granules, by spraying, atomizing, dusting, broadcasting or watering. The application forms depend entirely on the intended uses; they should in any case guarantee the finest possible dispersion of the active compounds according to the invention.

Aqueous application forms can be prepared from emulsion concentrates, pastes or wettable powders (spray powders, oil dispersions) by addition of water. To prepare emulsions, pastes or oil dispersions, the substances can be homogenized in water, as such or dissolved in an oil or solvent, by means of wetting agents, tackifiers, dispersants or emulsifiers. However, concentrates comprising active substance, wetting agent, tackifier, dispersant or emulsifier and possibly solvent or oil can also be prepared, which concentrates are suitable for dilution with water.

The concentrations of active compound in the ready-for-use preparations can be varied within relatively wide ranges. In general, they are between 0.0001 and 10%, preferably between 0.01 and 1%.

The active compounds can also be used with great success in the ultra low volume (ULV) process, it being possible to apply formulations with more than 95% by weight of active compound or even the active compound without additives.

Oils of various types, wetting agents, adjuvants, herbicides, fungicides, other pesticides and bactericides can be added to the active compounds, if need be also not until immediately before use (tank mix). These agents can be added to the compositions according to the invention in a weight ratio of 1:10 to 10:1.

The compositions according to the invention can, in the application form as fungicides, also be present together with other active compounds, e.g. with herbicides, insecticides, growth regulators, fungicides or also with fertilizers. On mixing the compounds I or the compositions comprising them in the application form as fungicides with other fungicides, in many cases an expansion of the fungicidal spectrum of activity is obtained.

The following list of fungicides, in conjunction with which the compounds according to the invention can be used, is intended to illustrate the possible combinations but does
acylalanines, such as benalaxyl, metalaxyl, ofurace or oxadixyl,
amine derivatives, such as aldimorph, dodine, dodemorph, fenpropimorph, fenpropidin, guazatine, iminoctadine, spiroxamine or tridemorph,
anilinopyrimidines, such as pyrimethanil, mepanipyrim or cyprodinyl,
antibiotics, such as cycloheximide, griseofulvin, kasugamycin, natamycin, polyoxin or streptomycin,
azoles, such as bitertanol, bromoconazole, cyproconazole, difenoconazole, dinitroconazole, epoxiconazole, fenbuconazole, fluquinconazole, flutriafol, flusilazole, hexaconazole, imazalil, metconazole, myclobutanil, penconazole, propiconazole, prochloraz, prothioconazole, tebuconazole, triadimefon, triadimenol, triflumizole or triticonazole,
dicarboximides, such as iprodione, myclozolin, procymidone or vinclozolin,
dithiocarbamates, such as ferbam, nabam, maneb, mancozeb, metam, metiram, propineb, polycarbamate, thiram, ziram or zineb,
heterocyclic compounds, such as anilazine, benomyl, boscalid, carbendazim, carboxin, oxycarboxin, cyazofamid, dazomet, dithianon, famoxadone, fenamidone, fenarimol, fuberidazole, flutolanil, furametpyr, isoprothiolane, mepronil, nuarimol, probenazole, proquinazid, pyrifenox, pyroquilon, quinoxyfen, silthiofam, thiabendazole, thifluzamide, thiophanate-methyl, tiadinil, tricyclazole or triforine,
copper fungicides, such as Bordeaux mixture, copper acetate, copper oxychloride or basic copper sulfate,
nitrophenyl derivatives, such as binapacryl, dinocap, dinobuton or nitrophthal-isopropyl,
phenylpyrroles, such as fenpiclonil or fludioxonil,
sulfur,
other fungicides, such as acibenzolar-5-methyl, benthiavalicarb, carpropamid, chlorothalonil, cyflufenamid, cymoxanil, dazomet, diclomezine, diclocymet, diethofencarb, edifenphos, ethaboxam, fenhexamid, fentin acetate, fenoxanil, ferimzone, fluazinam, fosetyl, fosetyl-aluminum, iprovalicarb, hexachlorobenzene, metrafenone, pencycuron, propamocarb, phthalide, tolclofos-methyl, quintozene or zoxamide,
strobilurins, such as azoxystrobin, dimoxystrobin, fluoxastrobin, kresoxim-methyl, metominostrobin, orysastrobin, picoxystrobin, pyraclostrobin or trifloxystrobin,
sulfenic acid derivatives, such as captafol, captan, dichlofluanid, folpet or tolylfluanid,
cinnamides and analogous compounds, such as dimethomorph, flumetover or flumorph.

### SYNTHESIS EXAMPLES

### Example 1

### Synthesis of 2-pyrazolyl-4-methoxy-5-(2,4,6-trifluorophenyl)-6-(2-methyl-butyl)pyrimidine [I-05]

### 1.1. 2-Methylthio-4-chloro-5-(2,4,6-trifluorophenyl)-6-(2-methylbutyl)pyrimidine

At about 10° C., 50 ml of 2-methylbutylmagnesium bromide solution (1 M in THF) was added to a mixture of 16.25 g (50 mmol) of 1-methylthio-4,6-dichloro-5-(2,4,6-trifluorophenyl)pyrimidine (WO 02/74753) and about 0.5 g of bisdiphenyl-phosphinoferrocenepalladium dichloride in 150 ml of toluene. The mixture was stirred at room temperature overnight (GC: about 35% of starting material), and a further 50 ml of 2-methylbutylmagnesium bromide solution (1 M in THF) were then added. The reaction mixture was then stirred at room temperature for 2.5 days. Sat. ammonium chloride solution was then added to the reaction mixture, and the mixture was extracted with methyl tert-butyl ether. The combined organic phases were concentrated and the residue was chromatographed on silica gel using cyclohexane/methyl tert-butyl ether 9:1. The combined product fractions were concentrated and the residue was chromatographed by preparative MPLC on RP-18 silica gel.

This gave 5.2 g (29%) of the title compound as a colorless oil.

¹H-NMR (CDCl₃, δ in ppm):

6.85 (t, 2H); 2.6 (s, 3H); 2.5 (dd, 1H); 2.25 (dd, 1H); 1.9 (m, 1H); 1.2 (m, 1H); 1.1 (m, 1H); 0.8 (m, 6H)

### 1.2. 2-Methylthio-4-methoxy-5-(2,4,6-trifluorophenyl)-6-(2-methylbutyl)pyrimidine

0.72 g (4 mmol) of sodium methoxide solution (30% strength in methanol) was added to a solution of 1.1 g (3 mmol) of 2-methylthio-4-chloro-5-(2,4,6-trifluoro-phenyl)-6-(2-methylbutyl)pyrimidine (Example 1.1.) in 20 ml of methanol, and the mixture was stirred at room temperature for 3.5 days. Sat. ammonium chloride solution was then added to the reaction mixture, and the mixture was extracted with methyl tert-butyl ether. The combined organic phases were concentrated and the residue was chromatographed by preparative MPLC on RP-18 silica gel. This gave 0.56 g (52%) of the title compound as a colorless resin.

MS: M⁺: 356

### 1.3. 2-Methylsulfonyl-4-methoxy-5-(2,4,6-trifluorophenyl)-6-(2-methylbutyl)pyrimidine

At 0° C., 0.9 g (3.93 mmol) of m-chloroperbenzoic acid was added to a solution of 0.56 g of 2-methylthio-4-methoxy-5-(2,4,6-trifluorophenyl)-6-(2-methylbutyl)-pyrimidine (Example 1.2.) in 20 ml of methylene chloride p. a. The mixture was stirred at room temperature overnight and the entire reaction mixture was then applied to a silica gel column. The product was eluted with cyclohexane/methyl tert-butyl ether 7:3, which gave 0.6 g (98%) of the title compound as a yellow oil.

¹H-NMR (CDCl₃, δ in ppm):

6.8 (t, 2H); 4.1 (s, 3H); 3.4 (s, 3H); 2.6 (dd, 1H); 2.4 (dd, 1H); 1.9 (m, 1H); 1.3 (m, 1H); 1.1 (m, 1H); 0.8 (m, 6H)

### 1.4. 2-Pyrazolyl-4-methoxy-5-(2,4,6-trifluorophenyl)-6-(2-methylbutyl)pyrimidine [I-05]

0.45 g (6.6 mmol) of pyrazole was added to a suspension of 0.18 g (7.4 mmol) of sodium hydride in 10 ml of tetrahydrofuran, and the mixture was stirred at room temperature for about 3 hours. 2.3 g (6 mmol) of 2-methylsulfonyl-4-methoxy-5-(2,4,6-trifluorophenyl)-6-(2-methylbutyl)pyrimidine (Example 1.3.) were then added, and the mixture was stirred at room temperature overnight. The reaction mixture was then diluted with sat. ammonium chloride solution and extracted with methyl tert-butyl ether. The combined organic phases were concentrated and the residue was purified by column chromatography using cyclohexane/methyl tert-butyl ether 7:3. This gave 0.21 g (9.3%) of the title compound as a colorless solid (m.p.=124° C.).

¹H-NMR (CDCl₃, δ in ppm):

8.65 (s, 1H); 7.85 (s, 1H); 6.8 (t, 2H); 6.5 (s, 1H); 4.05 (s, 3H); 2.6 (dd, 1H); 2.35 (dd, 1H); 2.0 (m, 1H); 1.3 (m, 1H); 1.1 (m, 1H); 0.8 (m, 6H)

### Example 2

### Synthesis of 2-triazolyl-4-methyl-5-(2,4,6-trifluorophenyl)-6-(2-methylbutyl)-pyrimidine [I-07]

### 2.1. 2-Methylthio-4-methyl-5-(2,4,6-trifluorophenyl)-6-chloropyrimidine

50 ml of methylmagnesium bromide solution (3 M in tetrahydrofuran) were added dropwise to a mixture of 32.5 g (0.1 mol) of 1-methylthio-4,6-dichloro-5-(2,4,6-trifluorophenyl)pyrimidine (WO 02/74753) and 0.5 g of bisdiphenyl-phosphinoferrocenepalladium dichloride in 150 ml of tetrahydrofuran p. a., during which the reaction temperature increased to about 40° C. The reaction mixture was stirred at room temperature overnight and sat. ammonium chloride solution was then added. The aqueous phase was extracted with methyl tert-butyl ether and the combined organic phases were concentrated. The residue was purified initially by silica gel chromatography using cyclohexane/methyl tert-butyl ether 9:1 and then by preparative MPLC on RP-18 silica gel. This gave 18.8 g (62%) of the title compound as a white solid.

¹H-NMR (CDCl₃, δ in ppm):

6.8 (t, 2H); 2.6 (s, 3H); 2.3 (s, 3H)

### 2.2. 2-Methylthio-4-methyl-5-(2,4,6-trifluorophenyl)-6-(2-methylbutyl)pyrimidine

At 50° C., 70 ml (0.035 mol) of a 0.5 M solution of 2-methylbutylmagnesium bromide (in tetrahydrofuran) were added to 9.1 g (30 mmol) of 2-methylthio-4-methyl-5-(2,4,6-trifluorophenyl)-6-chloropyrimidine (Example 2.1.) and about 200 mg of bisdiphenylphosphinoferrocenepalladium dichloride in 90 ml of toluene. After about 2 hours, an additional about 200 mg of bisdiphenyl-phosphinoferrocenepalladium dichloride and, a little at a time, a further 50 ml of a 0.5 M solution of 2-methylbutylmagnesium bromide (in tetrahydrofuran) were added. The reaction was monitored by HPLC.

The mixture was then hydrolyzed using sat. ammonium chloride solution and the aqueous phase was extracted with methyl tert-butyl ether. The combined organic phases were concentrated and the residue was purified by silica gel column chromatography using cyclohexane/methyl tert-butyl ether 9:1 and by preparative MPLC on RP-18 silica gel. This gave 5.9 g (58%) of the title compound as a colorless oil.

¹H-NMR (CDCl₃, δ in ppm):

6.8 (t, 2H); 2.6 (s, 3H); 2.45 (dd, 1H); 2.2 (s, 3H); 2.15 (dd, 1H); 1.9 (m, 1H); 1.25 (m, 1H); 1.05 (m, 1H); 0.8 (m, 6H)

### 2.3. 2-Methylsulfonyl-4-methyl-5-(2,4,6-trifluorophenyl)-6-(2-methylbutyl)pyrimidine

At 0° C., 2.8 g (12.3 mmol) of m-chloroperbenzoic acid (purity 77%) were added a little at a time to a solution of 1.9 g (5.6 mmol) of 2-methylthio-4-methyl-5-(2,4,6-trifluorophenyl)-6-(2-methylbutyl)pyrimidine (Example 2.2.) in 20 ml of methylene chloride p. a:, and the mixture was stirred at room temperature overnight. The reaction mixture was then applied directly to a silica gel column and eluted with cyclohexane/methyl tert-butyl ether 7:3. This gave 1.4 g (67%) of the title compound as a light-yellow oil.

¹H-NMR (CDCl₃, δ in ppm):

6.9 (t, 2H); 3.4 (s, 3H); 2.65 (dd, 1H); 2.45 (s, 3H); 2.4 (dd, 1H); 1.9 (m, 1H); 1.3 (m, 1H); 1.1 (m, 1H)

### 2.4. 2-(1,2,4-Triazolyl)-4-methyl-5-(2,4,6-trifluorophenyl)-6-(2-methylbutyl)pyrimidine [I-07]

0.15 g (2.2 mmol) of triazole was added to a mixture of 0.07 g (2.6 mmol) of sodium hydride in 10 ml of tetrahydrofuran, and the mixture was stirred at room temperature for about 3 hours. 0.38 g (1 mmol) of 2-methylsulfonyl-4-methyl-5-(2,4,6-trifluorophenyl)-6-(2-methylbutyl)pyrimidine (Example 2.3.) was then added, and the mixture was stirred at room temperature for about 4 hours. Sat. ammonium chloride solution was then added, and the aqueous phase was extracted with methyl tert-butyl ether. The combined organic phases were concentrated and the residue was purified by column chromatography using hexane/methyl tert-butyl ether 9:1. This gave 0.3 g (32%) of the title compound as a colorless oil.

¹H-NMR (CDCl₃, δ in ppm):

9.3 (s, 1H); 8.2 (s, 1H); 6.9 (t, 2H); 2.65 (dd, 1H); 2.45 (s, 3H); 2.4 (dd, 1H); 1.95 (m, 1H); 1.3 (m, 1H); 1.1 (m, 1H); 0.8 (m, 6H)

### Example 3

### Synthesis of 1-(1,2,4-triazolyl)-4-chloro-5-(2,4,6-trifluorophenyl)-6-cyclo-hexylpyrimidine [I-03]

### 3.1. 2-Methylthio-4-chloro-5-(2,4,6-trifluorophenyl)-6-cyclohexylpyrimidine

At room temperature, 50 ml of 2-methylbutylmagnesium bromide solution (1 M in THF) were added to a mixture of 16.25 g (50 mmol) of 1-methylthio-4,6-dichloro-5-(2,4,6-trifluorophenyl)pyrimidine (WO 02/74753) and about 0.5 g of bisdiphenyl-phosphinoferrocenepalladium dichloride in 150 ml of toluene, and the mixture was stirred at room temperature overnight.

Sat. ammonium chloride solution was then added to the reaction mixture, and the mixture was extracted with methyl tert-butyl ether. The combined organic phases were concentrated and the residue was chromatographed on silica gel using cyclohexane/methyl tert-butyl ether 20:1. The combined product fractions were concentrated and the residue was purified by preparative MPLC on RP-18 silica gel.

This gave 2.8 g (15%) of the title compound as a colorless oil.

¹H-NMR (CDCl₃, δ in ppm):

6.8 (t, 2H); 2.9 (m, 1H); 2.55 (s, 3H); 2.1 (d, broad, 2H); 1.9 (d, broad, 2H); 1.75 (d, broad, 1H); 1.7 (q, broad, 2H); 1.4 (m, 3H)

### 3.2. 2-Methylsulfonyl-4-chloro-5-(2,4,6-trifluorophenyl)-6-cyclohexylpyrimidine

At 0° C., 4.1 g (16.6 mmol) of m-chloroperbenzoic acid (about 75%) were added to 2.8 g (7.5 mmol) of 2-methylthio-4-chloro-5-(2,4,6-trifluorophenyl)-6-cyclohexyl-pyrimidine in 50 ml of methylene chloride p. a. The reaction mixture was stirred at room temperature overnight and then concentrated. The residue was taken up in ethyl acetate and the organic phase was extracted with sodium carbonate solution and water. The organic phase was concentrated and the residue was purified by column chromatography using cyclohexane/methyl tert-butyl ether 9:1. This gave 1.8 g (59%) of the title compound as a light-yellow oil.

¹H-NMR (CDCl₃, δ in ppm):

6.8 (t, 2H); 3.3 (s, 3H); 3.0 (m, 1H); 2.1 (d, broad, 2H); 1.9 (m, 2H); 1.7 (m, 1H); 1.65 (m, 2H); 1.4 (m, 3H)

### 3.3. 2-(1,2,4-Triazolyl)-4-chloro-5-(2,4,6-trifluorophenyl)-6-cyclohexylpyrimidine

0.15 g (2.2 mmol) of triazole was added to a mixture of 0.07 g (2.6 mmol) of sodium hydride in 10 ml of tetrahydrofuran, and the mixture was stirred at room temperature for about 3 hours. 0.40 g (1 mmol) of 2-methylsulfonyl-4-chloro-5-(2,4,6-trifluorophenyl)-6-cyclohexylpyrimidine (Example 3.2.) was then added, and the mixture was stirred at room temperature for about 4 hours. Sat. ammonium chloride solution was then added, and the aqueous phase was extracted with methyl tert-butyl ether. The combined organic phases were concentrated and the residue was purified by column chromatography using hexane/methyl tert-butyl ether 9:1. This gave 0.145 g (37%) of the title compound as a light-yellow oil.

¹H-NMR (CDCl₃, δ in ppm):

9.25 (s, 1H); 7.85 (s, 1H); 6.8 (t, 2H); 2.95 (m, 1H); 2.15 (m, 2H); 1.9 (m, 2H); 1.8 (m, 1H); 1.7 (m, 2H); 1.45 (m, 3H).

### Example 4

### Synthesis of 2-pyrazolyl-4-methyl-5-(2-fluoro-4-methylphenyl)-6-(3-methyl-but-1-enyl)pyrimidine [I-12]

### 4.1. 2-Hydroxy-4,6-dimethyl-5-bromopyrimidine

A mixture of 1.24 g (10 mmol) of 2-hydroxy-4,6-dimethylpyrimidine and 1.78 g (10 mmol) of N-bromosuccinimide in 20 ml of chloroform was stirred at room temperature for about 1 hour.

The reaction mixture was then concentrated and the residue was boiled with ethyl acetate. The hot suspension was filtered off with suction, the liquid phase was discarded and the residue was recrystallized from ethanol. This gave 0.8 g (39%) of the title compound as a light-brown solid.

¹H-NMR (DMSO-d₆; δ in ppm):

12.2 (s, 1H); 2.4 (s, 6H)

### 4.2. 2-Chloro-4,6-dimethyl-5-bromopyrimidine

4.52 g (30 mmol) of diethylaniline were added dropwise to a mixture of 6.1 g (30 mmol) of 2-hydroxy-4,6-dimethyl-5-bromopyrimidine (Example 4.1.) and 28 g (180 mmol) of phosphorous oxychloride. The reaction mixture was then stirred under reflux for about 8 hours. The reaction mixture was then hydrolyzed with ice-water and the aqueous phase was extracted with methylene chloride. The combined organic phases were washed with dilute hydrochloric acid and sodium bicarbonate solution, dried over magnesium sulfate and concentrated. The residue was purified by column chromatography using cyclohexane/methyl tert-butyl ether 9:1 and then by preparative MPLC on RP-18 silica gel. This gave 5.6 g (84%) of the title compound.

¹H-NMR (CDCl₃, δ in ppm):

2.65 (s, 6H)

### 4.3. 2-Pyrazolyl-4,6-dimethyl-5-bromopyrimidine

A little at a time, 1.5 g (22 mmol) of pyrazole were added to 0.7 g (26 mmol) of sodium hydride in 100 ml of tetrahydrofuran, and the mixture was stirred at room temperature for about 4 hours. 4.4 g (20 mmol) of 2-chloro-4,6-dimethyl-5-bromo-pyrimidine (Example 4.2.) were then added, and the mixture was stirred at room temperature overnight. The reaction mixture was diluted with ammonium chloride solution and extracted with methyl tert-butyl ether. The organic phase was concentrated and the residue was purified by preparative MPLC on RP-18 silica gel. This gave 1.1 g (22%) of the title compound.

¹H-NMR (CDCl₃, δ in ppm):

8.6 (s, broad, 1H); 7.8 (s, broad, 1H); 6.5 (s, broad, 1H); 2.7 (s, 6H)

### 4.4. 2-Pyrazolyl-4,6-dimethyl-5-(2-fluoro-4-methylphenyl)pyrimidine [I-11]

A mixture of 1.04 g (4 mmol) of 2-pyrazolyl-4,6-dimethyl-5-bromopyrimidine (Example 4.3.), 0.98 g (6 mmol) of 2-fluoro-4-methylphenylboronic acid, 0.52 g (6 mmol) of sodium bicarbonate and 1 spatula tip of tetrakistriphenylphosphine-palladium(0) in 5 ml of dimethoxyethane/water 1:1 was heated at reflux for about 3 hours. The reaction mixture was then diluted with water and extracted with methylene chloride. The organic phase was then concentrated and the residue was purified by column chromatography using cyclohexane/methyl tert-butyl ether mixtures and then by preparative MPLC on RP-18 silica gel. This gave 0.7 g (62%) of the title compound.

¹H-NMR (CDCl₃, δ in ppm):

8.7 (s, 1H); 7.8 (s, 1H); 7.1 (m, 3H); 6.5 (s, 1H); 2.45 (s, 3H); 2.35 (s, 6H)

### 4.5. 2-Pyrazolyl-4-methyl-5-(2-fluoro-4-methylphenyl)-6-(3-methylbut-1-enyl)-pyrimidine [I-12]

At −70° C., 0.8 ml (1.6 mmol) of lithium diisopropylamide solution (2 M in THF) was added dropwise to a mixture of 0.4 g (1.4 mmol) of 2-pyrazolyl-4,6-dimethyl-5-(2-fluoro-4-methylphenyl)pyrimidine (Example 4.4.) in 10 ml of tetrahydrofuran. The mixture was then stirred at −70° C. for about 30 min, and 0.1 g (1.4 mmol) of isobutyraldehyde were added dropwise using a syringe. The reaction mixture was stirred at −70° C. for about 2 hours and then allowed to warm to 0° C. The reaction mixture was then diluted with ammonium chloride solution and the aqueous phase was extracted with methyl tert-butyl ether. The combined organic phases were concentrated and the residue was purified by preparative MPLC on RP-18 silica gel. This gave 38 mg (8%) of the title compound.

¹H-NMR (CDCl₃, δ in ppm):

8.75 (s, 1H); 7.85 (s, 1H); 7.3 (m, 1H); 7.1 (m, 3H); 6.5 (s, 1H); 6.1 (d, 1H); 2.45 (s, 3H); 2.35 (s, 3H); 1.05 (d, 6H)

TABLE B

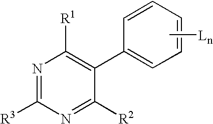

| | | | | | Physical data |
|---|---|---|---|---|---|
| Comp. | | | | | ¹H-NMR (δ in ppm; IR in cm⁻¹, |
| No. | R¹ | R² | R³ | (L)ₙ | m.p. in ° C.) |
| I-01 | 2-methyl- | chloro | [1,2,4]triazol- | 2,4,6-trifluoro | 9.3 (s, 1H); 8.2 (s, 1H); 6.9 (m, 1H); 2.7 |
| | butyl | | 1-yl | | (dd, 1H); 2.45 (dd, 1H); 2.0 (m, 1H); |
| | | | | | 1.3 (m, 1H); 1.15 (m, 1H); 0.8 (m, 6H) |
| I-02 | 2-methyl- | chloro | pyrazol-1-yl | 2,4,6-trifluoro | 8.65 (s, 1H); 7.9 (s, 1H); 6.85 (m, 2H); |
| | butyl | | | | 6.55 (s, 1H); 2.65 (dd, 1H); 2.4 (dd, |
| | | | | | 1H); 2.0 (m, 1H); 1.3 (m, 1H); 1.15 (m, |
| | | | | | 1H); 0.8 (m, 6H) |
| I-03 | cyclohexyl | chloro | [1,2,4]triazol- | 2,4,6-trifluoro | 9.25 (s, 1H); 7.85 (s, 1H); 6.8 (t, 2H); |
| | | | 1-yl | | 2.95 (m, 1H); 2.15 (m, 2H); 1.9 (m, 2H); |
| | | | | | 1.8 (m, 1H); 1.7 (m, 2H); 1.45 (m, 3H). |
| I-04 | 2-methyl- | methoxy | [1,2,4]triazol- | 2,4,6-trifluoro | 9.3 (s, 3H); 8.2 (s, 1H); 6.75 (m, 2H); |
| | butyl | | 1-yl | | 4.1 (s, 3H); 2.65 (dd, 1H); 2.4 (dd, 1H); |
| | | | | | 2.0 (m, 1H); 1.3 (m, 1H); 1.1 (m, 1H); |
| | | | | | 0.8 (m, 6H) |
| I-05 | 2-methyl- | methoxy | pyrazol-1-yl | 2,4,6-trifluoro | 8.65 (s, 1H); 7.85 (s, 1H); 6.8 (t, 2H); |
| | butyl | | | | 6.5 (s, 1H); 4.05 (s, 3H); 2.6 (dd, 1H); |
| | | | | | 2.35 (dd, 1H); 2.0 (m, 1H); 1.3 (m, 1H); |
| | | | | | 1.1 (m, 1H); 0.8 (m, 6H) |
| I-06 | cyclohexyl | chloro | pyrazol-1-yl | 2,4,6-trifluoro | 8.65 (s, 1H); 7.5 (s, 1H); 6.75 (m, 2H); |
| | | | | | 6.4 (s, 1H); 2.9 (m, 1H); 2.1 (m, 2H); |
| | | | | | 1.9 (m, 2H); 1.7 (m, 3H); 1.3 (m, 3H) |
| I-07 | 2-methyl- | methyl | [1,2,4]triazol- | 2,4,6-trifluoro | 9.3 (s, 1H); 8.2 (s, 1H); 6.9 (t, 2H); 2.65 |
| | butyl | | 1-yl | | (dd, 1H); 2.45 (s, 3H); 2.4 (dd, 1H); |
| | | | | | 1.95 (m, 1H); 1.3 (m, 1H); 1.1 (m, 1H); |
| | | | | | 0.8 (m, 6H) |
| I-08 | but-1-en- | methyl | pyrazol-1-yl | 2,4,6-trifluoro | 8.55 (s, 1H); 7.45 (s, 1H); 6.75 (m, |
| | 4-yl | | | | 2H); 6.4 (s, 1H); 6.0 (m, 1H); 5.15 (d, |
| | | | | | 1H); 5.05 (d, 1H); 3.1 (t, 2H); 2.7 (m, |
| | | | | | 2H); 2.4 (s, 3H) |
| I-09 | but-1-en- | methyl | [1,2,4]triazol- | 2,4,6-trifluoro | 9.2 (s, 1H); 7.8 (s, 1H); 6.75 (m, 2H); |
| | 4-yl | | 1-yl | | 5.95 (m, 1H); 5.15 (d, 1H); 5.05(d, 1H); |
| | | | | | 3.15 (t, 2H); 2.7 (m, 2H); 2.45 (s, 3H) |
| I-10 | 2-methyl- | methyl | pyrazol-1-yl | 2,4,6-trifluoro | 8.7 (s, 1H); 7.85 (s, 1H); 6.85 (m, 1H); |
| | butyl | | | | 6.5 (s, 1H); 2.6 (dd, 1H); 2.35 (dd, 1H); |
| | | | | | 1.95 (m, 1H); 1.3 (m, 1H); 1.1 (m, 1H); |
| | | | | | 0.8 (m, 6H) |
| I-11 | methyl | methyl | pyrazol-1-yl | 2-fluoro-4- | 8.7 (s, 1H); 7.8 (s, 1H); 7.1 (m, 3H); 6.5 |
| | | | | methyl | (s, 1H); 2.45 (s, 3H); 2.35 (s, 6H) |
| I-12 | 3-methyl- | methyl | pyrazol-1-yl | 2-fluoro-4- | 8.6 (s, 1H); 7.8 (s, 1H); 7.15 (s, 1H); |
| | but-1-enyl | | | methyl | 7.1 (m, 3H); 6.5 (s, 1H); 6.1 (d, 1H); |
| | | | | | 2.6 (m, 2H); 2.45 (s, 3H); 2.35 (s, 3H); |
| | | | | | 1.05 (d, 6H) |
| I-13 | 2-hydroxy- | methyl | pyrazol-1-yl | 2-fluoro-4- | 93-102 |
| | 3-methyl- | | | methyl | |
| | butyl | | | | |
| I-14 | methyl | methyl | pyrazol-1-yl | 2,4-difluoro | 99-102 |
| I-15 | 3-methyl- | methyl | 1,2,4-triazol- | 2-fluoro-4- | 1.0 (d, 6H), 2.3 (s, 3H), 2.0 (s, 3H), |
| | but-1-enyl | | 1-yl | methyl | 6.1 (d, 1H), 7.1-7.2 (m, 3H), 7.25- |
| | | | | | 7.32 (m, 1H), 8.2 (s, 1H), 9.4 (s, 1H) |
| I-16 | 2-methyl- | methyl | pyrazol-1-yl | 2,4-difluoro | 0.80-0.85 (m, 6H), 2.20-2.30 (m, 1H), |
| | propyl | | | | 2.40 (s, 3H), 2.45-2.55 (m, 2H), 6.5 |
| | | | | | (t, 1H), 6.85-7.05 (m, 2H), 7.15-7.20 |
| | | | | | (m, 1H), 7.75 (s, 1H), 8.65 (s, 1H) |
| I-17 | 2-methyl- | methyl | 1,2,4-triazol- | 2,4-difluoro | 77-83 |
| | propyl | | 1-yl | | |
| I-18 | 2-methyl- | methyl | 1,2,3-triazol- | 2,4-difluoro | 0.80-0.95 (m, 6H), 2.1-2.25 (m, 1H), |
| | propyl | | 1-yl | | 2.40 (s, 3H), 2.5-2.65 (m, 2H), 6.80- |
| | | | | | 7.05 (m, 2H), 7.15-7.25 (m, 1H), 7.75 |
| | | | | | (s, 1H), 7.90 (s, 1H), 8.70 (s, 1H) |
| | | | | | Diastereomers (1:1) |
| I-19 | 2-methyl- | chloro | 1,2,3-triazol- | 2,4,6-trifluoro | 52-56 |
| | butyl | | 1-yl | | |
| I-20 | 2-methyl- | chloro | 3-cyano- | 2,4,6-trifluoro | 54-57 |
| | butyl | | 1,2,4-triazol- | | |
| | | | 1-yl | | |
| I-21 | 2-methyl- | chloro | 7-amino- | 2,4,6-trifluoro | 51-55 |
| | butyl | | indazol-1-yl | | |
| I-22 | 2-methyl- | chloro | 3-amino- | 2,4,6-trifluoro | 53-57 |
| | butyl | | pyrazol-1-yl | | |
| I-23 | 2-hydroxy- | methyl | pyrazol-1-yl | 2-fluoro | 0.75-0.9 (m, 6H), 1.20-1.40 (m, 2H), |
| | 3-methyl- | | | | 2.4 (s, 3H), 2.70-2.90 (m, 1H), 6.5 (d, |
| | butyl | | | | 1H), 7.15-7.35 (m, 2H), 7.40-7.50 (m, |
| | | | | | 1H), 7.80 (d, 1H), 8.6 (d, 1CH) |
| | | | | | atropisomers. |
| I-24 | 2-hydroxy- | methyl | pyrazol-1-yl | 2,4-difluoro | 0.8-0.95 (m, 6H), 1.6-1.75 (m, 1H), |
| | 3-methyl- | | | | 2.55-2.80 (m, 2H), 3.75-3.95 (m, 1H), |
| | butyl | | | | 6.45 (s, 1H), 6.9-7.3 (m, 3H), 7.75 (s, |
| | | | | | 1H), 8.6 (s, 1H) |
| I-25 | methyl | methyl | pyrazol-1-yl | 2,4-dichloro | 1.55 (s, 6H), 6.9-7.5 (m, 6H) |
| I-26 | 2-methyl- | methyl | pyrazol-1-yl | 2,5-dichloro | 0.8-0.9 (m, 6H), 2.2-2.5 (m, 6H), 6.5 |
| | propyl | | | | (m, 1H), 7.15 (s, 1H), 7.35 (d, 1H), |
| | | | | | 7.5 (s, 1H), 7.8 (d, 1H), 8.7 (d, 1H) |
| I-27 | 2-methyl- | methyl | 1,2,4-triazol- | 2,5-dichloro | 0.8-0.9 (m, 6H), 2.25-2.5 (m, 6H,), |
| | propyl | | 1-yl | | 7.2 (s, 1H), 7.4 (d, 1H), 7.5 (d, |
| | | | | | 1H) 8.2 (s, 1H), 9.25 (s, 1H) |
| I-28 | 2-methyl- | methyl | pyrazol-1-yl | 2-fluoro | 0.8-0.9 (m, 6H), 2.2-2.5 (m, 6H), 6.5 |
| | propyl | | | | (s, 1H), 7.15-7.5 (m, 4H), 7.75 (s, |
| | | | | | 1H), 8.7 (s, 1H) |
| I-29 | 2-methyl- | methyl | 1,2,4-triazol- | 2-fluoro | 0.7-0.9 (m, 6H), 2.1-2.5 (m, 6H,), 7.1- |
| | propyl | | 1-yl | | 7.5 (m, 4H), 8.2 (s, 1H). 9.30 (s, 1H) |
| I-30 | 2-methyl- | methyl | 1,2,3-triazol- | 2-fluoro | 0.80-0.90 (m, 6H), 2.20-2.30 (m, 1H), |
| | propyl | | 1-yl | | 2.45 (s, 3H), 2.50-2.65 (m, 2H), 7.1- |
| | | | | | 7.5 (m, 4H), 7.75 (s, 1H), 8.70 (s, 1H) |
| I-31 | 2-methyl- | methyl | 1,2,3-triazol- | 2,4,6-trifluoro | 39-43 |
| | butyl | | 1-yl | | |
| I-32 | 3-methyl- | methyl | pyrazol-1-yl | 2,4-difluoro | 0.80-0.90 (m, 6H), 2.30 (s, 3H), 2.35- |
| | but-1-enyl | | | | 2.50 (m, 1H), 6.00 (d, 1H), 6.50 (t, |
| | | | | | 1H), 6.90-7.30 (m, 4H), 7.80 (d, 1H), |
| | | | | | 8.75 (d, 1H) |
| I-33 | 2-methyl- | methyl | pyrazol-1-yl | 2,4-dichloro | 0.80 (d, 3H), 0.90 (d, 3H), 2.20-2.500 |
| | propyl | | | | (m, 6H), 6.5 (s, 1H), 7.1 (d, 1H), 7.4 |
| | | | | | (d, 1H), 7.6 (s, 1H), 7.85 (s, 1H), 8.65 |
| | | | | | (s, 1H) |
| I-34 | 2-methyl- | methyl | 1,2,4-triazol- | 2,4-dichloro | 98-102 |
| | propyl | | yl | | |
| I-35 | 2-methyl- | methyl | 1,2,3-triazol- | 2,4-dichloro | 0.80 (d, 3H), 0.85 (d, 3H), 2.20-2.30 |
| | propyl | | 1-yl | | (m, 2H), 2.35 (s, 3H), 2.50-2.55 (m, |
| | | | | | 1H), 7.15 (d, 1H), 7.40 (d, 1H), 7.60 |
| | | | | | (s, 1H), 7.85 (s, 1H), 8.65 (s, 1H) |
| I-36 | methyl | chloro | 1,2,4-triazol- | 2,4,6-trifluoro | 2.5 (s, 3H), 6.85 (t, 2H), 8.2 (s, 1H), |
| | | | 1-yl | | 9.3 (s, 1H) |

Examples of the Activity Against Harmful Fungi

The fungicidal action of the compounds of the formula I was demonstrated by the following experiments:

The active compounds were formulated, separately or jointly, as a 10% emulsion in a mixture of 70% by weight of cyclohexanone, 20% by weight of Nekanil® LN (Lutensol® AP6, wetting agent having emulsifying and dispersant action based on ethoxylated alkylphenols) and 10% by weight of Wettol® EM (nonionic emulsifier based on ethoxylated castor oil) and diluted with water to give the desired concentration.

### Use Example 1: Activity Against Early Blight of Tomato Caused by Altemaria solani

Leaves of potted plants of the tomato cultivar “Large Fruited St. Pierre” were sprayed to runoff point with an aqueous suspension having the concentration of active compounds stated below. The suspension or emulsion was prepared from a stock solution comprising 10% of active compound in a mixture consisting of 85% of cyclohexanone and 5% of emulsifier. The next day, the leaves were infected with an aqueous spore suspension of Alternaria solani in a 2% biomalt solution having a density of 0.17×10⁶ spores/ml. The plants were then placed in a water-vapor-saturated chamber at 20-22° C. After 5 days, the blight on the untreated but infected control plants had developed to such an extent that the infection could be determined visually in %.

In this experiment, plants which had been treated with 250 ppm of active compound I-01, I-02 or I-04 showed an infection of 0 to 5%, whereas the untreated plants were 100% infected.

Use Example 2: Activity Against Gray Mold on Bell Pepper Leaves Caused by Botrytis cinerea

Bell pepper seedlings of the cultivar “Neusiedler Ideal Elite” were, after they had 4-5 well-developed leaves, sprayed to runoff point with an aqueous suspension of the concentration of active compounds stated below. The suspension or emulsion was prepared from a stock solution comprising 10% of active compound in a mixture consisting of 85% of cyclohexanone and 5% of emulsifier. The next day, the treated plants were inoculated with a spore suspension of Botrytis cinerea which contained 1.7×10⁶ spores/ml in a 2% aqueous biomalt solution. The test plants were then placed in a climatized chamber at 22-24° C. and high atmospheric humidity. After 5 days, the extent of the fungal infection on the leaves could be determined visually in %.

In this experiment, plants which had been treated with 250 ppm of active compound I-01, I-02 or I-04 showed an infection of 0 to 5%, whereas the untreated plants were 100% infected.

1. A pyrimidine of the formula I 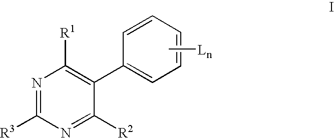
in which the index and the substituents are as defined below:
n is an integer from 1 to 5;
L is halogen, cyano, nitro, cyanato (OCN), C₁-C₈-alkyl, C₂-C₁₀-alkenyl, C₂-C₁₀-alkynyl, C₁-C₆-alkoxy, C₂-C₁₀-alkenyloxy, C₂-C₁₀-alkynyloxy, C₃-C₆-cycloalkyl, C₃-C₆-cycloalkenyl, C₃-C₆-cycloalkoxy, C₃-C₆-cycloalkenyloxy, —C(═S)—N(A′)A, —C(═O)-A, —C(═O)—O-A, —C(═O)—N(A′)A, C(A′)(═N-OA), N(A′)A, N(A′)-C(═O)-A, N(A″)—C(═O)—N(A′)A, S(═O)ₘ-A, S(═O)ₘ—OA or S(═O)ₘ—N(A′)A;
m is 0, 1 or 2;
A,A′, A″ independently of one another are hydrogen, C₁-C₆-alkyl, C₂-C₆-alkenyl, C₂-C₆-alkynyl, C₃-C₈-cycloalkyl, C₃-C₈-cycloalkenyl, where the organic radicals may be partially or fully halogenated or may be substituted by cyano or C₁-C₄-alkoxy, or A and A′ together with the atoms to which they are attached are a five- or six-membered saturated, partially unsaturated or aromatic heterocycle which contains one to four heteroatoms from the group consisting of O, N and S;
R¹ is C₁-C₁₀-alkyl, C₂-C₁₀-alkenyl, C₂-C₁₀-alkynyl, C₃-C₁₂-cycloalkyl, C₃-C₁₀-cycloalkenyl;
R² is halogen, cyano, C₁-C₄-alkyl, C₂-C₄-alkenyl, C₂-C₄-alkynyl, C₁-C₄-alkoxy, C₃-C₄-alkenyloxy or C₃-C₄-alkynyloxy;
R³ is a five- or six-membered saturated, partially unsaturated or aromatic mono- or bicyclic heterocycle which contains one to four heteroatoms from the group consisting of O, N and S,
where the aliphatic, alicyclic or aromatic groups of the radical definitions of L, R¹, R² and/or R³ for their part may be partially or fully halogenated or may carry one to four groups R^{a}:
R^{a} is halogen, cyano, C₁-C₈-alkyl, C₂-C₁₀-alkenyl, C₂-C₁₀-alkynyl, C₁-C₆-alkoxy, C₂-C₁₀-alkenyloxy, C₂-C₁₀-alkynyloxy, OH, SH, two vicinal groups R^{a} may be (═O) or (═S), C₃-C₆-cycloalkyl, C₃-C₆-cycloalkenyl, C₃-C₆-cycloalkoxy, C₃-C₆-cycloalkenyloxy, —C(═O)-A, —C(═O)—O-A, —C(═O)—N(A′)A, C(A′)(═N-OA), N(A′)A, N(A′)-C(═O)-A, N(A″)—C(═O)—N(A′)A, S(═O)ₘ-A, S(═O)ₘO-A or S(═O)ₘ—N(A′)A, where m, A, A′, A″ are as defined above and where the aliphatic, alicyclic or aromatic groups for their part may be partially or fully halogenated or may carry one to three groups R^{b}, where R^{b} has the same meaning as R^{a}.

2. A pyrimidine as claimed in claim 1, in which the index and the substituents are as defined below:
L is halogen, cyano, C₁-C₈-alkyl, C₂-C₁₀-alkenyl, C₂-C₁₀-alkynyl, C₁-C₆-alkoxy, C₂-C₁₀-alkenyloxy, C₂-C₁₀-alkynyloxy, —C(═O)—O-A, N(A′)-C(═O)-A or S(═O)ₘ-A;
m is 0, 1 or 2;
A,A′, A″ independently of one another are hydrogen, C₁-C₆-alkyl, C₂-C₆-alkenyl, C₂-C₆-alkynyl, C₃-C₈-cycloalkyl, where the organic radicals may be partially or fully halogenated or A and A′ together with the atoms to which they are attached are a partially unsaturated or aromatic heterocycle which contains one to four heteroatoms from the group consisting of O, N and S;
R¹ is C₁-C₁₀-alkyl, C₂-C₁₀-alkenyl, C₂-C₁₀-alkynyl, C₃-C₁₂-cycloalkyl, C₃-C₁₀-cycloalkenyl;
R² is C₁-C₄-alkyl, cyano or chlorine,
where the aliphatic, alicyclic or aromatic groups of the radical definitions of L, R¹ and/or R³ for their part may be partially or fully halogenated or may carry one to four groups R^{a}:
R^{a} is halogen, cyano, C₁-C₈-alkyl, C₂-C₁₀-alkenyl, C₂-C₁₀-alkynyl, C₁-C₆-alkoxy, C₂-C₁₀-alkenyloxy, C₂-C₁₀-alkynyloxy, C₃-C₆-cycloalkyl, C₃-C₆-cycloalkenyl, C₃-C₆-cycloalkoxy, C₃-C₆-cycloalkenyloxy, —C(═O)-A, —C(═O)—O-A, —C(═O)—N(A′)A, C(A′)(═N-OA), N(A′)A, N(A′)-C(═O)-A, N(A″)—C(═O)—N(A′)A, S(═O)ₘ-A, S(═O)ₘ—O-A or S(═O)ₘ—N(A′)A.

3. A pyrimidine as claimed in claim 1, in which R³ is pyrrolyl, pyrazolyl, imidazolyl, 1,2,3-triazolyl, 1,2,4-triazolyl, tetrazolyl, oxazolyl, isoxazolyl, 1,3,4-oxadiazolyl, furanyl, thiophenyl, thiazolyl, isothiazolyl, pyridinyl, pyrimidinyl, pyrazinyl, pyridazinyl, 1,2,3-triazinyl, 1,2,4-triazinyl, pyrrolidinyl, piperidinyl, hexahydroazepinyl or dihydropyridinyl, where the heterocycle may be attached to the pyrimidine ring via carbon or nitrogen and may carry up to three substituents R^{a}:
R^{a} is halogen, cyano, C₁-C₈-alkyl, C₂-C₁₀-alkenyl, C₂-C₁₀-alkynyl, C₁-C₆-alkoxy, C₂-C₁₀-alkenyloxy, C₂-C₁₀-alkynyloxy, OH, SH, two vicinal groups R^{a} may be (═O) or (═S), C₃-C₆-cycloalkyl, C₃-C₆-cycloalkenyl, C₃-C₆-cycloalkoxy, C₃-C₆-cycloalkenyloxy, —C(═O)-A, —C(═O)—O-A, —C(═O)—N(A′)A, C(A′)(═N-OA), N(A′)A, N(A′)-C(═O)-A, N(A″)—C(═O)—N(A′)A, S(═O)ₘ-A, S(═O)ₘ—O-A or S(═O)ₘ—N(A′)A.

4. A pyrimidine as claimed in claim 1, in which R³ is pyrazol-1-yl, [1,2,4]-triazol-1-yl, pyridin-2-yl, pyrimidin-2-yl, pyridazin-3-yl, pyrrolidin-2-on-1-yl, piperidin-2-on-1-yl, hexahydro-2H-azepin-2-on-1-yl, pyrrolidin-2-thion-1-yl, piperidin-2-thion-1-yl, hexahydro-2H-azepin-2-thion-1-yl, 1,2-dihydropyridin-2-on-1-yl.

5. A pyrimidine as claimed in claim 1, in which R² is methyl, chlorine or ethyl.

6. A pyrimidine as claimed in any of claims 1 to 5, in which the phenyl group substituted by Lₙ is the group B 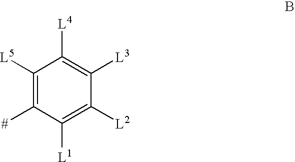
where # is the point of attachment to the pyrimidine skeleton and
L¹ is fluorine, chlorine, CH₃ or CF₃;
L²,L⁴ independently of one another are hydrogen, CH₃ or fluorine;
L³ is hydrogen, fluorine, chlorine, bromine, cyano, CH₃, SCH₃, OCH₃, SO₂CH₃, CO—NH₂, CO—NHCH₃, CO—NHC₂H₅, CO—N(CH₃)₂, NH—C(═O)CH₃, N(CH₃)—C(═O)CH₃ or COOCH₃ and
L⁵ is hydrogen, fluorine, chlorine or CH₃.

7. A process for preparing pyrimidines of the formula I as claimed in claim 1, where R³ is a nitrogen-containing heterocycle attached via nitrogen, which comprises reacting a compound of the formula III, 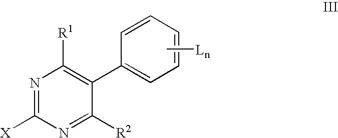
in which the substituents Lₙ, R¹ and R² are as defined in claim 1 and X is halogen, C₁-C₆-alkoxy, C₁-C₆-alkylthio, C₁-C₆-alkylsulfoxyl or C₁-C₆-alkylsulfenyl, with a heterocycle of the formula R³—H (IV), if appropriate in the presence of a base.

8. An intermediate of the formula III 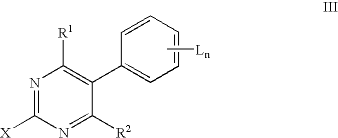
in which the substituent R¹ is as defined in claim 1, Lₙ is as defined in claim 2, X is as defined in claim 7 and R² is cyano, C₁-C₄-alkyl, C₂-C₄-alkenyl, C₂-C₄-alkynyl, C₁-C₄-alkoxy, C₃-C₄-alkenyloxy or C₃-C₄-alkynyloxy, where the alkyl, alkenyl and alkynyl radicals of R² may be substituted by halogen, cyano, nitro, C₁-C₂-alkoxy or C₁-C₄-alkoxycarbonyl.

9. A pesticidal composition, which comprises a solid or liquid carrier and a compound of the formula I as claimed in claim 1.

10. A method for controlling phytopathogenic harmful fungi, which comprises treating the fungi or the materials, plants, the soil or seeds to be protected against fungal attack with an effective amount of a compound of the formula I as claimed in claim 1.
